(19) United States
(12) Patent Application Publication
(10) Pub. No.: US 2026/0102733 A1
(43) Pub. Date: Apr. 16, 2026

(54) VACUUM SYSTEM

(71) Applicant: Honeywell International Inc., Charlotte, NC (US)

(72) Inventors: Rebecca Kamire, Buffalo Grove, IL (US); Mark Triezenberg, Westmont, IL (US); David Gray, Des Plaines, IL (US); David Loeffelholz, Long Beach, CA (US); Meghan Pipitone, Scottsdale, AZ (US); Stephen Yates, South Barrington, IL (US); Emir Rahislic, Chicago, IL (US); Eric D. Pope, Glendale, AZ (US)

(21) Appl. No.: 19/351,083

(22) Filed: Oct. 6, 2025

Related U.S. Application Data
(63) Continuation of application No. 19/087,168, filed on Mar. 21, 2025.
(60) Provisional application No. 63/705,858, filed on Oct. 10, 2024.
(60) Provisional application No. 63/705,844, filed on Oct. 10, 2024.

(51) Int. Cl. B01D 53/14; B01D 53/18; B01D 53/26; C07C 1/12

(52) U.S. Cl. CPC B01D 53/1412; B01D 53/1425; B01D 53/1475; B01D 53/185; B01D 53/26; C07C 1/12; B01D 2257/504; B01D 2257/80

(57) ABSTRACT

A vacuum system includes a first compression stage including a steam jet ejector and one or more subsequent compression stages downstream of the first compression stage. The first compression stage is configured to compress a fluid stream received from a vessel, such as a stripper, to a first pressure and maintain a vacuum on the vessel less than about 50 torr. Each subsequent compression stage is configured to further compress the fluid stream, such that the subsequent compression stages compress the fluid stream to a final pressure.

### CROSS-REFERENCE TO RELATED APPLICATIONS

This application is a continuation of U.S. patent application Ser. No. 19/087,168, filed Mar. 21, 2025 and entitled “CONTAMINANT REMOVAL SYSTEM USING LIQUID SORBENT-BASED PACKED BED CONTACTOR,” which claims the benefit of U.S. Provisional Patent Application 63/705,844, filed Oct. 10, 2024 and entitled “VACUUM SYSTEM,” and U.S. Provisional Patent Application 63/705,858, filed Oct. 10, 2024 and entitled “CONTAMINANT REMOVAL SYSTEM USING LIQUID SORBENT-BASED PACKED BED CONTACTOR,” the entire contents of each of which are incorporated herein by reference.

### TECHNICAL FIELD

The present disclosure relates to systems and techniques for removing contaminants.

### BACKGROUND

An environmental control system (ECS) of a structure, such as a building or vehicle, may remove carbon dioxide expelled by occupants of an environment, such as a room or cabin, to maintain comfort and safety. In some instances, the carbon dioxide may be absorbed from the environment by a liquid sorbent and desorbed from the liquid sorbent for discharge from the structure.

### SUMMARY

The disclosure describes systems and techniques for removing contaminants, such as carbon dioxide, using liquid sorbents.

In some examples, the systems and techniques remove contaminants using liquid sorbent-based packed bed contactors. The contaminants are absorbed from a cabin air stream into the liquid sorbent at a scrubber and desorbed from the liquid sorbent at a stripper. Rather than absorb and desorb the contaminants across a membrane, which may introduce mass transfer resistance and have reduced life at higher pressure drops, at least one of the scrubber or the stripper is a packed bed contactor that provides a high surface area for mass transfer of contaminants into or from the liquid sorbent. When used as a stripper, the packed bed contactor may be capable of operating at higher temperatures and gas flow rates, thereby increasing a rate of desorption of contaminants from the liquid sorbent. For example, a membrane stripper may increase liquid pressure with flow rate, reducing a life of the membrane, and the membrane may have a lower maximum temperature limit than packing media of a packed bed stripper. In contrast, a pressure drop through the packing media of the packed bed stripper may be substantially lower than a pressure drop across the membrane of a membrane stripper, enabling deeper vacuum at the liquid/gas interface of the packing media for increased driving force and faster stripping.

Water in the cabin air stream may be removed prior to reaching the scrubber using a condensing heat exchanger having a low pressure drop, which can be later added back to a clean air stream discharged by the scrubber. Flow of the liquid sorbent through the scrubber and/or stripper may be individually controlled through recirculation to achieve particular absorption and desorption rates, respectively. The packed bed contactors and condensing heat exchanger may utilize gravity to drive flow of liquid sorbent over packing material of the packed bed contactor or flow of condensed water from tubes of the condensing heat exchanger. In these various ways, contaminant removal systems described herein may operate with reduced size, weight, and power and increased reliability compared to contaminant reduction systems that transfer contaminants between segregated phases across membranes.

In some examples, the disclosure describes a contaminant removal system that includes a scrubber, a stripper, a liquid sorbent circuit, and a conditioning assembly. The scrubber is configured to absorb one or more contaminants from an air stream into a liquid sorbent. The stripper is configured to desorb the one or more contaminants from the liquid sorbent. The liquid sorbent circuit is configured to circulate the liquid sorbent between the scrubber and the stripper. The conditioning assembly is configured to maintain a vacuum on the stripper. At least the stripper includes a packed bed contactor.

In some examples, the disclosure describes a contaminant desorption system that includes a stripper, a liquid sorbent pump, and a vacuum pump. The stripper is configured to desorb one or more contaminants from a liquid sorbent. The liquid sorbent pump is configured to receive the liquid sorbent from the stripper. The vacuum pump is configured to maintain a vacuum on the stripper and pressurize a contaminant stream from the stripper. The stripper includes a packed bed contactor.

In some examples, the disclosure describes a method for removing one or more contaminants from an air stream. The method includes absorbing, by a scrubber, the one or more contaminants from the air stream into a liquid sorbent, desorbing, by a stripper, the one or more contaminants from the liquid sorbent, circulating, by a liquid sorbent circuit, the liquid sorbent between the scrubber and the stripper, and maintaining, by a conditioning assembly, a vacuum on the stripper. At least the stripper includes a packed bed contactor.

In some examples, the systems and techniques absorb contaminants, such as carbon dioxide, using a liquid sorbent and desorb the contaminants using a medium vacuum. The contaminants are absorbed into the liquid sorbent at a scrubber and desorbed from the liquid sorbent at a stripper. The driving force of contaminants from the liquid sorbent increases as the pressure in the stripper decreases, such that operating the stripper at medium vacuum may increase desorption and enable the stripper to be operated at lower temperatures.

To achieve a medium vacuum (e.g., less than 50 torr) in the stripper, a vacuum system downstream of the stripper includes a steam jet ejector that is capable of higher volumes and lower maintenance operation than other vacuum sources, such as vacuum pumps. The steam jet ejector is part of a first compression stage that compresses a mixed fluid stream (e.g., carbon dioxide and water) received from the stripper. One or more subsequent compression stages downstream of the first compression stage further remove water and compress the fluid stream to a desired pressure, such as greater than about 70 kPa. The subsequent compression stage or stages may include compression components, such as liquid ring pumps, that operate quietly and with lower maintenance than dry vacuum pumps, such as rotary vane vacuum pumps. In this way, a contaminant removal system may be capable of operating with increased efficiency, increased reliability, and reduced complexity compared to contaminant removal systems that use only vacuum pumps to generate vacuum for desorption and compress desorbed contaminants.

In some examples, the disclosure describes a vacuum system that includes a first compression stage configured to compress a fluid stream received from a vessel to a first pressure, and one or more subsequent compression stages downstream of the first compression stage configured to compress the fluid stream to a final pressure. The first compression stage includes a steam jet ejector and may be configured to maintain a vacuum on the vessel less than about 50 torr. Each subsequent compression stage is configured to further compress the fluid stream.

In some examples, the disclosure describes a contaminant removal system that includes a scrubber, a stripper, and the vacuum system described above. The scrubber is configured to absorb one or more contaminants from an air stream using a liquid sorbent. The stripper is configured to desorb the one or more contaminants from the liquid sorbent into a contaminant stream, which the vacuum system compresses.

In some examples, the disclosure describes a method for compressing a fluid stream from a vacuum that includes receiving, by a first compression stage, the fluid stream from a vessel and maintaining, by the first compression stage, the vacuum on the vessel less than about 50 torr. The method further includes compressing, by the first compression stage, the fluid stream to a first pressure and compressing, by one or more subsequent compression stages downstream of the first compression stage, the fluid stream to a final pressure.

### BRIEF DESCRIPTION OF THE FIGURES

The details of one or more examples are set forth in the accompanying drawings and the description below. Other features, objects, and advantages will be apparent from the description and drawings, and from the claims.

FIG. 1A is a block diagram illustrating an example contaminant removal system for removing contaminants using one or more liquid sorbent-based packed bed contactors.

FIG. 1B is an example flowchart of a method for removing contaminants using one or more liquid sorbent-based packed bed contactors.

FIG. 2 is a schematic diagram illustrating an example contaminant removal system for removing contaminants using one or more liquid sorbent-based packed bed contactors.

FIG. 3A is a schematic diagram illustrating a liquid sorbent circuit of an example contaminant removal system for removing contaminants using one or more liquid sorbent-based packed bed contactors.

FIG. 3B is a schematic diagram illustrating an example contaminant desorption system for desorbing contaminants from a liquid sorbent using a liquid sorbent-based packet bed contactor.

FIG. 4A is a block diagram illustrating an example contaminant removal system that includes a vacuum system for desorbing contaminants.

FIG. 4B is an example flowchart of a method for compressing a fluid stream from a vacuum.

FIG. 5 is a schematic diagram illustrating an example contaminant removal system that includes a vacuum system for desorbing contaminants that includes a steam jet ejector in a first compression stage and a water-sealed liquid ring pump in a second compression stage.

FIG. 6 is a schematic diagram illustrating an example vacuum system for contaminant desorption that includes steam jet ejectors in both first and second compression stages.

FIG. 7 is a schematic diagram illustrating an example vacuum system for contaminant desorption that includes a steam ejector in a first compression stage and an ionic liquid-sealed liquid ring pump in a second compression stage.

### DETAILED DESCRIPTION

In some aspects, the disclosure describes systems and techniques for removing contaminants, such as carbon dioxide, from an air stream using liquid sorbents with one or more packed-bed contactors. Contaminant reduction systems described herein may be utilized as part of an environmental control system (ECS), such as in spacecraft, aircraft, watercraft, and the like. In some examples, contaminant reduction systems may be used in an ECS of a resource-limited gravity environment, such as aircraft, watercraft, or non-Earth gravity environments (e.g., lunar or Martian). Such resource-limited environments may be particularly suited for a contaminant reduction system that includes components that utilize gravity for liquid distribution, separation, and/or collection, thereby operating with reduced size, weight, and power and increased reliability compared to contaminant reduction systems that transfer contaminants between segregated phases across membranes.

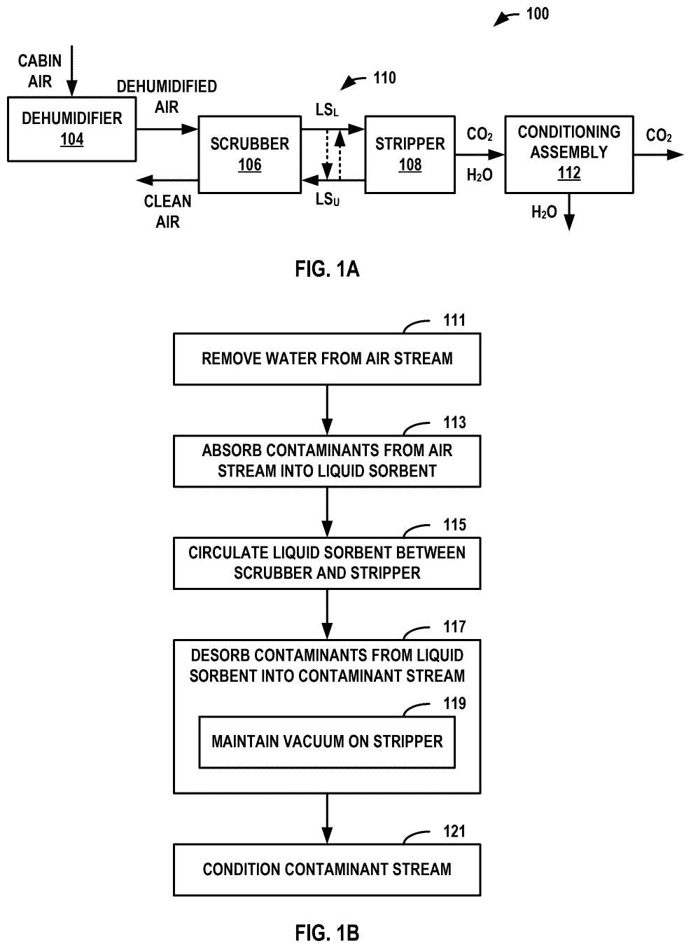

FIG. 1A is a block diagram illustrating an example contaminant removal system 100 for removing contaminant from an air stream using at least one liquid sorbent-based packed bed contactor. Contaminant removal system 100 is configured to remove at least a portion of the contaminants in the air stream using one or more liquid sorbents. A liquid sorbent may include any liquid configured to absorb and desorb a gaseous species, such as an ionic liquid sorbent. Liquid sorbents may be used with contactors that contact an air stream with or draw an air stream from the liquid sorbent across one or more hydrophobic porous membranes.

Contaminant removal system 100 includes one or more dehumidifiers 104, one or more scrubbers 106 downstream of dehumidifier 104, one or more strippers 108 downstream of scrubber 106, a liquid sorbent circuit 110 between scrubber 106 and stripper 108, and a conditioning assembly 112 downstream of stripper 108.

Dehumidifier 104 is configured to remove water from an air stream (“CABIN AIR”) and, in some instances, return at least a portion of the removed water back to another air stream (“CLEAN AIR”) once contaminants are removed from the air stream.

Scrubber 106 is configured to absorb one or more contaminants from the dehumidified air stream (“DEHUMIDIFIED AIR”) from dehumidifier 104 into a liquid sorbent. The one or more contaminants may include carbon dioxide (CO₂) and/or other contaminants, such as water vapor (H₂O). Stripper 108 is configured to desorb the one or more contaminants from the liquid sorbent and discharge an air stream (“CLEAN AIR”) having a lower concentration of contaminants than the received air stream. Liquid sorbent circuit 110 is configured to circulate the liquid sorbent between scrubber 106 and stripper 108. Loaded liquid sorbent (LS_{L}) that includes a relatively high concentration of contaminants is transferred from scrubber 106 to stripper 108, and unloaded liquid sorbent (LS_{U}) that includes a relatively low concentration of contaminants is transferred from stripper 108 to scrubber 106.

Conditioning assembly 112 is configured to maintain a vacuum on stripper 108 and condition a contaminant stream (“CO₂, H₂O”) discharged from stripper 108, such as by pressurizing and removing water from the contaminant stream.

When operating in a microgravity environment, dehumidifier 104, scrubber 106, and stripper 108 may include membrane dehumidifiers and contactors that use membranes as an interface between two air streams or an air stream and a liquid sorbent. However, such membranes may have relatively high pressure drop requirements, may be more susceptible to damage at high vacuum, and may be costly to replace. For example, an efficiency of stripper 108 may be improved by operating stripper 108 at a higher temperature or higher vacuum. However, a high liquid-to-gas pressure drop across stripper 108 and/or high temperature at stripper 108 may reduce a durability of the membrane, resulting in either lower efficiency or higher maintenance costs.

To improve an efficiency and/or reliability of contaminant removal system 100 in a gravity environment, any or all of dehumidifier 104, scrubber 106, and/or stripper 108 may include a respective dehumidifier or contactor that does not include membranes. A gravity environment may enable distribution, separations, and/or collection of different phases, such as water from dehumidified air at dehumidifier 104 and/or liquid sorbent from contaminants at stripper 108, that may not be possible or efficient in a microgravity environment in which phases do not separate or remain separate due to gravity.

At least one of scrubber 106 or stripper 108 may be a packed bed contactor. Packed bed contactors are configured to absorb contaminants into and/or desorb contaminants from a liquid sorbent at a gas/liquid interface on a surface of packing media. For example, a packed bed contactor may be a vertical column filled with packing material that provides a large surface area for contact between gas in the air stream and the liquid sorbent. The liquid sorbent may be introduced near a top of the column, such that the liquid sorbent flows over the packing media to the bottom of the column. The packing media may be mechanically robust compared to membranes and may provide a substantially lower pressure drop between the liquid sorbent and the gas. While the packing media may have lower effective surface area compared to membranes, a mass transfer rate of the contaminants may be higher due to absence of the membrane as an impediment to migration of the contaminants.

For use of a packed bed contactor as scrubber 106, as the gas flows through the packed bed, the gas comes into contact with the liquid sorbent flowing over the packing material. The contaminants in the gas phase dissolve into the liquid sorbent due to differences in partial pressures and solubility. The cleaned gas exits the column, and the loaded liquid sorbent is collected at the bottom. For use of a packed bed contactor as scrubber 106, the contaminants are transferred from the liquid sorbent to a gas phase due to differences in partial pressures and vapor pressures. The unloaded liquid sorbent exits the column, while the contaminant stream is discharged to conditioning assembly 112.

Stripper 108 may particularly benefit from use of a packed bed contactor, as stripper 108 may be operated at higher temperatures and/or deeper vacuum compared to a membrane contactor. For example, a rate of desorption from the liquid sorbent may be related to a partial pressure gradient between the liquid sorbent and gas phase, which may be increased by increasing temperature of the liquid sorbent and/or decreasing a pressure of the gas phase. The packing media in a packed bed contactor may be better able to handle increased temperature and pressure drops than membranes of a membrane contactor, enabling stripper 108 to operate at higher temperatures and/or vacuums.

In the example of FIG. 1A, liquid sorbent circuit 110 may include one or more recirculation subcircuits configured to recirculate liquid sorbent through scrubber 106 and/or stripper 108. For example, scrubber 106 and stripper 108 may absorb and desorb contaminants at different rates, such that a flow rate of liquid sorbent through each of scrubber 106 and stripper 108 may be controlled to achieve a desired rate. Such varying flow rates may be possible due to lower pressure drop, and corresponding higher flow rates, accommodated by packed bed contactors.

In some examples, dehumidifier 104 may be a condensing heat exchanger that is configured to dehumidify the air stream prior to discharging the air stream to scrubber 106. A condensing heat exchanger is configured to cool an air stream using a coolant and condense at least a portion of a condensable constituent, such as water, from the air stream. As a result, an amount of water vapor in the air stream, and thus an amount of water absorbed by the liquid sorbent at scrubber 106 and subsequently desorbed by stripper 108, may be reduced. A pressure drop through the condensing heat exchanger may be relatively low compared to a membrane dehumidifier, as tubes may provide substantially less resistance to flow than membranes. In some examples, contaminant removal system 100 may also include a humidifier (not shown), such as a membrane humidifier or an evaporator, configured to rehumidify the clean air stream discharged by scrubber 106 using water removed by the condensing heat exchanger.

In some examples, contaminant removal system 100 may be configured to remove carbon dioxide for use in generating methane. A contaminant stream desorbed by and discharged from stripper 108 may be at a relatively low pressure, and may include contaminants, such as water vapor, which may inhibit a Sabatier rection. Conditioning assembly 112 may be configured to condition the contaminant stream desorbed from stripper 108 by separating various contaminants other than carbon dioxide, such as water, such that the contaminant stream includes substantially only carbon dioxide, and bringing the carbon dioxide up to conditions for a Sabatier reaction. For example, the conditions of the Sabatier reaction, in combination with flow conditions and a composition of a catalyst, may be selected for a relatively high carbon dioxide conversion and methane selectivity. Such conditions may include a temperature between about 250 degrees Celsius (° C.) and about 450° C., and a pressure between about 55 kilopascals (kPa) and about 100 kPa.

FIG. 1B is an example flowchart of a method for removing contaminants using one or more liquid sorbent-based packed bed contactors. FIG. 1B will be described with respect to FIG. 1A; however, other systems may be used to perform the method of FIG. 1B, including for fluid streams other than contaminant streams.

The method of FIG. 1B includes removing water from a cabin air stream (111). For example, dehumidifier 104 may be maintained at particular conditions, such as flow rate of the cabin air stream or temperature of cooling fluid in dehumidifier 104, to facilitate removal of water to another fluid stream, such as by controlling a blower or a chiller. These particular conditions may be based on a particular measured or anticipated humidity of the cabin air stream.

The method of FIG. 1B includes absorbing one or more contaminants from a cabin air stream into a liquid sorbent (113). For example, liquid sorbent circuit 110 may maintain scrubber 106 at particular conditions, such as temperature and flow rate of liquid sorbent, to facilitate transfer of the one or more contaminants into the liquid sorbent, such as by controlling a pump to circulate the liquid sorbent between scrubber 106 and stripper 108, controlling a heat exchanger to cool the liquid sorbent prior to entry into scrubber 106, and/or controlling a heat exchanger to recover a portion of heat from liquid sorbent discharged from stripper 108. These particular conditions may be based on a particular measured or anticipated composition of the cabin air stream. The method of FIG. 1B includes circulating the liquid sorbent, including the one or more contaminants, between scrubber 106 and stripper 108 (115).

The method of FIG. 1B includes desorbing the one or more contaminants from the liquid sorbent (117). For example, liquid sorbent circuit 110 and/or conditioning assembly 112 may maintain stripper 108 at particular conditions, such as temperature and flow rate of liquid sorbent and vacuum at stripper 108, to facilitate transfer of the one or more contaminants from the liquid sorbent, such as by controlling conditioning assembly 112 to maintain a vacuum at stripper 108, controlling a pressure of the liquid sorbent to maintain a pressure head on a liquid sorbent pump, or controlling a heater to heat the liquid sorbent prior to entry into stripper 108. These particular conditions may be based on a particular measured or anticipated composition of the cabin air stream. In the example of FIG. 1B, desorbing contaminants from the liquid sorbent includes maintaining a vacuum on stripper 108 (119).

The method of FIG. 1B includes further conditioning the contaminant stream (121). For example, conditioning assembly 112 may be operated at particular conditions, such as stream flow rate or pressure, to maintain the vacuum on stripper 108, compress the contaminant stream received from stripper 108 to a desired pressure, such as about 55 kPa to about 100 kPa for a Sabatier reaction, and remove water from the contaminant stream.

In some examples, liquid sorbent circuit 110 and/or conditioning assembly 112 may maintain stripper 108 at a relatively high temperature and/or vacuum, such as less than 10 torr. For example, stripper 108 may be a packed bed contactor, which may be more resistant to temperatures and pressure drops than membrane contactors.

In some examples, the method of FIG. 1B may include generating methane from carbon dioxide in the contaminant stream (not shown). For example, a Sabatier reactor may be configured to receive the contaminant stream from conditioning assembly 112 and hydrogen gas from another source, and generate methane from the carbon dioxide and the hydrogen gas.

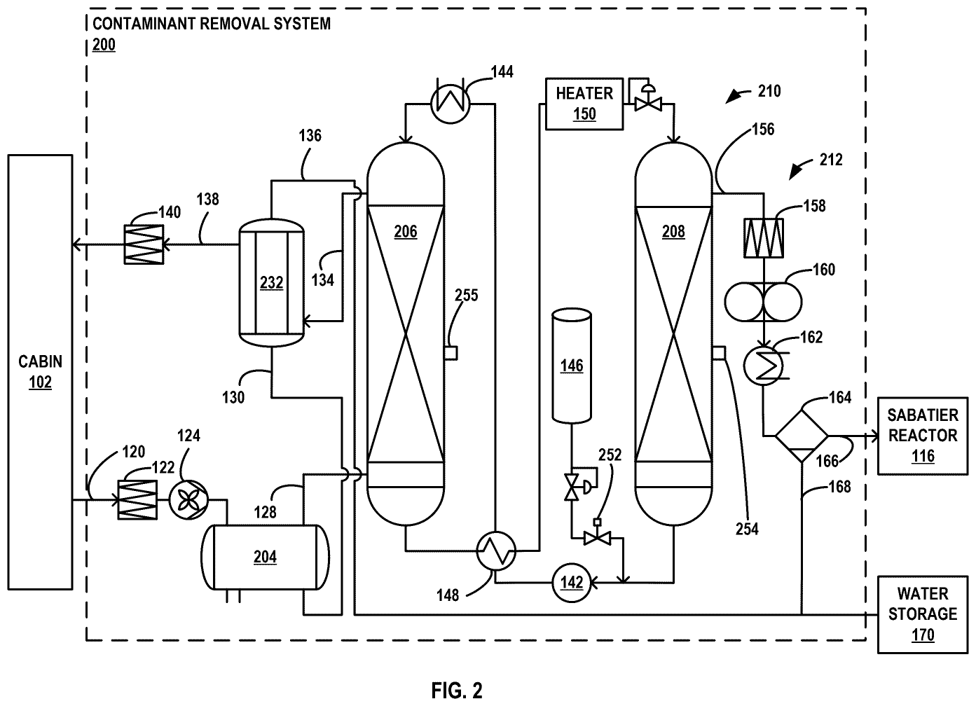

FIG. 2 is a schematic diagram illustrating an example contaminant removal system 200 for removing contaminants using one or more liquid sorbent-based packed bed contactors. Contaminant removal system 200 includes a cabin air circuit (not labeled) configured to circulate cabin air between cabin 102 and scrubber 206 via a condensing heat exchanger 204 and a humidifier 232. In the example of FIG. 2, a cabin air stream 120 includes a filter 122 configured to remove particulates from cabin air stream 120 prior to entry into scrubber 206 and a blower 124 configured to draw cabin air into scrubber 106, while rehumidified air stream 138 includes a filter 140 configured to remove any leaked liquid sorbent and/or further filter clean air from rehumidified air stream 138 prior to entry into cabin 102.

Contaminant removal system 200 includes condensing heat exchanger 204 configured to capture humidity from cabin air stream 120 that may otherwise be absorbed by the liquid sorbent at scrubber 206. For example, condensing heat exchanger 204 may be positioned between cabin 102 and scrubber 206, such that cabin air received by scrubber 206 may include a lower humidity than cabin air received by contaminant removal system 200 from cabin 102. Condensing heat exchanger 204 may include any heat exchanger configured to condense and separate a condensable from a fluid stream using a cooling fluid. In some examples, condensing heat exchanger 204 may use gravity to at least partially drive separation or collection of the condensable, such as by including a liquid outlet at a bottom of condensing heat exchanger to collect condensate.

On one side, condensing heat exchanger 204 may be configured to receive cabin air stream 120 as a feed gas stream, discharge cabin air in a dehumidified air stream 128 to scrubber 206 having a lower humidity, and discharge condensate in a condensate stream 130. While shown in FIG. 2 as being discharged to humidifier 232, in other examples, such as examples that do not include humidifier 232, condensate stream 130 may be directed elsewhere, such as water storage 170. On an opposite side, condensing heat exchanger 204 may be configured to receive a cooling fluid, such as chilled water, that absorbs heat from cabin air stream 120 to condense at least a portion of water vapor or other condensables in cabin air stream 120.

By removing water prior to going through scrubber 206, a reduced amount of water may be absorbed into the liquid sorbent at scrubber 206 and, correspondingly, removed by stripper 208 through evaporative cooling. This water removal by condensing heat exchanger 204 may permit smaller sizing of scrubber 206 and/or stripper 208, and/or a smaller load on a liquid sorbent pump 142 due to reduced volume of liquid sorbent, and/or may reduce an amount of power for a heater 150 due to reduced evaporative cooling. Additionally, condensing heat exchanger 204 may have a relatively low pressure drop for cabin air stream 120 compared to other dehumidifiers, such as membrane dehumidifiers, thereby enabling a smaller or lower power blower 124.

In the example of FIG. 2, contaminant removal system 200 also includes humidifier 232 configured to recover humidity captured from cabin air stream 120 into clean air stream 134 that may otherwise be discharged or stored. For example, humidifier 232 may be positioned between scrubber 206 and cabin 102, such that clean air received by cabin 102 may include a lower humidity than cabin air received by contaminant removal system 200 from cabin 102. By recapturing humidity removed from cabin air prior to entry of the cabin air from cabin air stream 120 into scrubber 206, a greater amount of humidity may be preserved.

Humidifier 232 may include any humidifier configured to increase a humidity of an air stream, such as a membrane humidifier (as shown in FIG. 2), an evaporator, or a spray-based humidifier. In examples in which humidifier 232 is a membrane humidifier, on one side of a membrane, humidifier 232 may be configured to receive a clean air stream 134 from scrubber 206, receive water from across the membrane into a gas phase, and discharge clean air to rehumidified air stream 138 having a higher humidity. On an opposite side, humidifier 232 may receive water from condensate stream 130 and discharge a spent condensate stream 136 to water storage 170. In examples in which humidifier 232 is an evaporator, humidifier 232 may be configured to receive water from condensate stream 130, receive clean air stream 134 from scrubber 206, evaporate a portion of the water, and discharge clean air to rehumidified air stream 138 having a higher humidity.

Contaminant removal system 200 is configured to remove at least a portion of the contaminants in the air stream using one or more liquid sorbents. A liquid sorbent may include any liquid configured to absorb and desorb a gaseous species. Liquid sorbents may be water soluble, hygroscopic (i.e., capable of absorbing moisture from the air), capable of absorbing or desorbing contaminants in response to a change in solubility driven by a change in temperature, and/or capable of releasing water by evaporation, such as by elevating the temperature or reducing the water partial pressure. In some examples, the liquid sorbent may be an ionic liquid sorbent. Ionic liquid sorbents may be salts that are generally comprised of an anion and an organic cation. These salts may be liquid at their temperature of use, have effectively zero vapor pressure, be generally nontoxic, and/or have sufficient stability to resist deterioration. In some examples, ionic liquid sorbents may contain relatively large organic cations and any of a variety of anions, which may be tailored to obtain desired characteristics, such as characteristics that improve absorption of the particular contaminant under operating conditions of contaminant removal system 200. A variety of ionic liquid sorbents may be used including, but not limited to, imidazolium salts, such as 1-ethyl-3-methylimidazolium (EMIM) acetate (Ac).

In contaminant removal system 200, the liquid sorbent is dissolved in water to form a liquid sorbent mixture. A concentration of liquid sorbent in the liquid sorbent mixture may be sufficiently high to remove a particular or set of contaminants and sufficiently low that the liquid sorbent remains in solution through operating ranges (e.g., temperature range, pH range) and/or maintains a low viscosity for maintaining high mass transfer. In some examples, the liquid sorbent mixture may further include a dissolved promoter. The promoter may be configured to increase a rate of removal of a contaminant, such as water or carbon dioxide, from an air stream. For example, the promoter may be configured to reduce a viscosity of the liquid sorbent, change a pH of the liquid sorbent, increase a thermal stability of the liquid sorbent, increase a capacity of the liquid sorbent for the contaminant, or increase an absorption rate of the contaminant into the liquid sorbent. Absorption of the contaminants by the liquid sorbent may be determined by a concentration of the contaminants in the corresponding air stream.

Liquid sorbents may be used with contactors that contact an air stream with or draw an air stream from the liquid sorbent. In some instances, contactors may be membrane contactors that maintain substantially single phases (liquid and gas) on opposite sides of one or more hydrophobic porous membranes. While these hydrophobic porous membranes may have high surface areas, the membranes may have a relatively low overall rate of mass transfer of the contaminants. For example, mass transfer of contaminants across a membrane at a stripper may include migration of contaminants to the membrane, desorption of the contaminants from the liquid sorbent, and migration of the desorbed contaminants across the membrane. Migration of the desorbed contaminants across the membrane may be a rate limiting step.

In the example of FIG. 2, each of scrubber 206 and stripper 208 is a packed bed contactor. Rather than flow air on a first side of the membrane and flow liquid sorbent on a second, opposite side, packed bed contactors may flow liquid over a packed media that is in direct contact with the air. A packed bed contactor may include a vessel, at least a portion of which is filled with packing media forming a contact surface area. The high surface area of the packing enables a high mass transfer of contaminant gases, such as carbon dioxide and water, into and/or from the liquid sorbent using a relatively small system volume and weight. Flow of the liquid sorbent over the packing media and collection of the liquid sorbent in the vessel may be driven by gravity, such that the liquid sorbent may not require segregation from a gas phase, as in a membrane contactor. Various parameters of the packing media, such as the shape, size, and material, may be selected such that the packed bed contactor has a low pressure drop for the liquid sorbent and/or an air stream, has a high degree of mixing (e.g., via turbulence created by the packing material) and liquid distribution, has a desired residence time of the liquid sorbent, has a desired void fraction for a gas phase, and/or has a desired surface area for contact between the liquid sorbent and the gas phase. A variety of materials may be used for the packing media including, but not limited to, ceramics, metals, plastics, and any other material that may be capable of operating at a desired operating temperature. A variety of shapes may be used for the packing material including, but not limited to, loose or random packing media, such as Raschig rings, saddles, Pall rings, ceramic balls, or hollow tubes; fixed packing media, such as fins, rigid grids, or wire meshes; structured packing media, such as honeycomb structures or corrugated sheets; or other packing media having high surface area. In some examples, the packing media may be further coated to improve wettability.

Scrubber 206 is configured to absorb one or more contaminants from cabin air stream 120 into the liquid sorbent and discharge clean air stream 134 to cabin 102. Clean air stream 134 has a lower concentration of contaminants than cabin air stream 120. For example, clean air stream 134 may have a concentration of carbon dioxide that is about 25% to about 99% less than a concentration of carbon dioxide in cabin air stream 120. Scrubber 206 is configured to receive cabin air from dehumidified air stream 128 that includes contaminants from cabin 102. Contaminants may become absorbed by the liquid sorbent on the packing media. As a result, clean air from clean air stream 134 discharged from scrubber 206 may have a lower concentration of contaminants than cabin air from dehumidified air stream 128 received by scrubber 206. Scrubber 206 is configured to discharge clean air stream 134 to cabin 102 via humidifier 232. Scrubber 206 is configured to receive unloaded liquid sorbent, such as from a liquid sorbent storage vessel 146. The unloaded liquid sorbent may flow through packing media in scrubber 206 and absorb carbon dioxide and other gaseous contaminants from cabin air. As a result, the loaded liquid sorbent discharged from scrubber 206 may have a higher concentration of contaminants than the unloaded liquid sorbent received by scrubber 206. Scrubber 206 may discharge the loaded liquid sorbent containing the contaminants to stripper 208.

Stripper 208 is configured to desorb the contaminants, such as carbon dioxide, from the liquid sorbent into contaminant stream 156. Stripper 208 is configured to receive loaded liquid sorbent from scrubber 206 and desorb contaminants from the loaded liquid sorbent on the packing media. As a result, unloaded liquid sorbent discharged from stripper 208 may have a lower concentration of contaminants than the loaded liquid sorbent received by stripper 208. Stripper 208 is configured to discharge the contaminants in contaminant stream 156. Contaminant stream 156 may be continuously removed from stripper 208 to assist migration of the contaminants from the loaded liquid sorbent into contaminant stream 156.

While packed bed contactors may include packing media that provides some amount of agitation, such agitation may be limited by passive flow control through the packed bed. To further increase mass transfer into and/or from the liquid sorbent, scrubber 206 and/or stripper 208 may include one or more ultrasonic (or ultrasound) transducers 255 and 254, respectively, that increase agitation of the fluid and decrease diffusion limits of mass transfer based on diffusion of the contaminant within the fluid to facilitate mass transfer between at the interface between the liquid sorbent and the gas phase.

Without being limited to any particular theory, ultrasonic transducers 255, 254 may enhance absorption or desorption of contaminants into or from a liquid sorbent by generating high-frequency sound waves. These sound waves create rapid pressure fluctuations, leading to several physical effects that facilitate desorption. For example, the high-frequency sound waves may generate microscopic bubbles in the liquid sorbent that collapse to produce intense localized pressure and temperature spikes, which can disrupt the gas-liquid interface and promote gas release in stripper 208 or gas absorption in scrubber 206. The sound waves also may induce bulk movement of the liquid sorbent, which may bring gas molecules in the liquid sorbent closer to the gas-liquid interface during desorption or move gas molecules away from the gas-liquid interface during absorption, enhancing mass transfer and mixing within the packed bed. In the case of stripper 208, ultrasonic transducer 254 may further cause heating of the liquid sorbent, thereby enhancing desorption and/or reducing a power consumption at heater 150. While ultrasonic transducers 254, 255 may be used with any type of packing media, in some examples, packed bed contactors incorporating ultrasonic transducers 254, 255 may include a fixed packing media to reduce agitation of the packing media.

Ultrasonic transducers 254, 255 may be positioned at a variety of locations on or within scrubber 206 or stripper 208 including mounting on external walls as shown in FIG. 2, directly immersing within the liquid sorbent. While only a single ultrasonic transducer 254, 255 is shown for stripper 208 and scrubber 206, respectively, any number of ultrasonic transducers may be used. For example, ultrasonic transducers may be positioned at different heights and circumferential locations. Various operating parameters, such as frequency and power level, of ultrasonic transducers 254, 255 may be selected based on various properties of the liquid sorbent. For example, the frequency and power level may be adjusted to achieve effective cavitation and mixing without causing excessive heating or damage to the packing material. Properties of the liquid sorbent may influence operation of the ultrasonic transducers 254, 255 may include, but are not limited to, viscosity of the liquid sorbent, density of the liquid sorbent, surface tension of the liquid sorbent, or other properties of the liquid sorbent that may affect cavitation and/or mixing.

While both scrubber 206 and stripper 208 are illustrated as being packed bed contactors, in some examples, only one of scrubber 206 or stripper 208 is a packed bed contactor, and the other of scrubber 206 and stripper 208 is another type of contactor, such as a membrane contactor. For example, stripper 208 may include a packed bed contactor, as stripper 208 may operate at temperatures and pressures for which a packed bed contactor may be particularly useful, while scrubber 206 may include a membrane contactor. A membrane contactor may include a cylindrical module filled with parallel or woven hollow porous fibers forming a hydrophobic porous membrane. The high surface area of the hollow fiber membrane contactors enables a high mass transfer of contaminant gases, such as carbon dioxide and water, into the respective liquid sorbent using a relatively small system volume and weight. The material of the hollow fibers can be selected such that the liquid sorbent does not wet the pores, and the trans-membrane pressure is kept sufficiently low to prevent pore penetration. Fiber materials may include, but are not limited to, hydrophobic materials such as polypropylene, polyvinylidene fluoride, polysulfone, polyimide, polytetrafluoroethylene (PTFE), and the like. In some examples, a coating may be applied to reduce liquid flow through the pores. Coatings that may be used include, but are not limited to, PTFE, a crosslinked siloxane, perfluorinated polymers, functionalized nanoparticles, and the like to prevent liquid flow through the pores.

Contaminant removal system 200 includes liquid sorbent circuit 210 configured to circulate liquid sorbent between scrubber 206 and stripper 208 and control various parameters of the liquid sorbent, such as temperature, pressure, and flow rate. For example, a liquid sorbent pump 142 may pump unloaded liquid sorbent from stripper 208 into scrubber 206. Unloaded liquid sorbent may include unused liquid sorbent free of contaminants or regenerated liquid sorbent having a lower concentration of contaminants than the loaded liquid sorbent. Liquid sorbent storage vessel 146 may store liquid sorbent, such as in a relatively cool state, and in some instances, may provide additional pressure head for liquid sorbent pump 142.

Liquid sorbent circuit 210 may include one or more components for controlling a pressure of the liquid sorbent. Liquid sorbent pump 142 may be configured to draw liquid sorbent from stripper 208. Stripper 208 may be run at a relatively low vacuum, such as a deep vacuum enabled by the robust packing media used in stripper 208. However, such high vacuum may reduce a net positive suction head (NPSH) available at an inlet to liquid sorbent pump 142. Liquid sorbent circuit 210 may be configured to maintain an adequate pressure head at an inlet to liquid sorbent pump 142 by controlling a pressure of the liquid sorbent and/or stripper 208.

In some examples, liquid sorbent pump 142 may be configured to operate with a low NPSH. For example, liquid sorbent pump 142 may be a positive displacement pump or other continuous flow displacement-based, rather than velocity-based or discontinuous flow, pump. In some examples, liquid sorbent circuit 210 may include a liquid sorbent supply valve 252 configured to discharge liquid sorbent from liquid sorbent storage vessel 146 into the liquid sorbent stream upstream of liquid sorbent pump 142. For example, a liquid level in liquid sorbent storage vessel 146 may be controlled to raise the available NPSH to liquid sorbent pump 142. In some examples, liquid sorbent circuit 210 may include a stripper repressurization valve upstream of stripper 208.

Liquid sorbent circuit 210 may include one or more components for controlling a temperature of the liquid sorbent. In some examples, the unloaded liquid sorbent may be cooled by a regenerative heat exchanger 148 and/or a chiller 144 prior to entry into scrubber 206. Heat exchanger 148 is configured to exchange heat between a relatively hot unloaded liquid sorbent from stripper 208 and a relatively cool loaded liquid sorbent from scrubber 206. Chiller 144 is configured to receive the unloaded liquid sorbent from stripper 208, cool the unloaded liquid sorbent, and discharge the cooled, unloaded liquid sorbent to scrubber 206. Liquid sorbent circuit 210 may include one or more heaters 150 upstream of stripper 208. Heaters 150 may be configured to heat the liquid sorbent prior to entry into stripper 208 to increase desorption of contaminants from the liquid sorbent.

In the example of FIG. 2, contaminant removal system 200 may include one or more systems or components, such as a conditioning assembly 212, configured to further process contaminant stream 156, such that carbon dioxide may be isolated and pressurized for reaction in Sabatier reactor 116. Contaminant removal system 200 includes Sabatier reactor 116 configured to generate hydrocarbons using carbon dioxide removed by scrubber 206. Sabatier reactor 116 may require a water concentration of less than 10% to react hydrogen with carbon dioxide. However, in a life support application, a large amount of water may be present in cabin air stream 120.

Conditioning assembly 212 includes a vacuum pump 160, a condenser 162, and a water separator 164 configured to pressurize contaminant stream 156 and remove water from the compressed contaminant stream. For example, for carbon dioxide removed from stripper 208 to be reacted efficiently by Sabatier reactor 116, vacuum pump 160, condenser 162, and water separator 164 may pressurize contaminant stream 156 to a moderate pressure and remove nearly all water from contaminant stream 156. However, this pressure may be substantially lower than a pressure at which carbon dioxide would otherwise be stored at. In some examples, contaminant removal system 200 includes a filter 158 configured to remove entrained liquid sorbent from contaminant stream 156.

Vacuum pump 160 is configured to pressurize contaminant stream 156 and draw a vacuum on stripper 208. Vacuum pump 160 is configured to pressurize the carbon dioxide to a reaction pressure. In some examples, the reaction pressure is less than about 100 kilopascals (kPa). For example, vacuum pump 160 may be configured to operate at a pressure between about 50 kPa and about 100 kPa. A variety of vacuum pumps may be used for vacuum pump 160 including, but not limited to, centrifugal compressors, positive displacement compressors, and the like. Condenser 162 is configured to cool contaminant stream 156 and condense water from contaminant stream 156. For example, condenser 162 may be coupled to a cooling medium system or other cooling system that circulates a cooling medium to cool contaminant stream 156. A variety of condensers may be used for condenser 162 including, but not limited to, shell and tube heat exchangers, plate-fin, surface coolers, heat pipes, thermoelectric devices, cooling jackets, and the like. Water separator 164 is positioned upstream of Sabatier reactor 116 and is configured to separate desorbed water from the carbon dioxide. Water separator 164 may be configured to remove water from contaminant stream 156, discharge a purified contaminant stream 166 to Sabatier reactor 116, and discharge water condensate stream 168 to water storage 170. A variety of water separators may be used for water separator 164 including, but not limited to, static phase separators, capillary phase separator, membrane phase separators, centrifugal/rotary separators, and the like.

Sabatier reactor 116 is configured to generate methane from the carbon dioxide and, to a lesser degree, carbon monoxide. Sabatier reactor 116 includes a catalyst configured to increase a reaction rate of the Sabatier reaction. In some examples, a structure of the catalyst may be configured to further increase thermal and/or mass transfer of reactants and products. For example, Sabatier reactor 116 may include a catalyst in the form of any of a mesh, a packed bed, a microchannel grid, or other structure or combination of structures having a high surface area, high thermal conductivity, and/or high reactant throughput. Catalysts that may be used include, but are not limited to, nickel, ruthenium, rhodium, and the like, alone or on a support, such as aluminum oxide. In some examples, Sabatier reactor 116 is configured to operate at a relatively high carbon dioxide conversion (e.g., >70%) and methane selectivity (e.g., >80%) at relatively low temperatures and pressures. For example, the catalyst may be configured to achieve such high carbon dioxide conversion and methane selectivity at temperatures less than about 450° C. and/or pressures less than about 100 kPa.

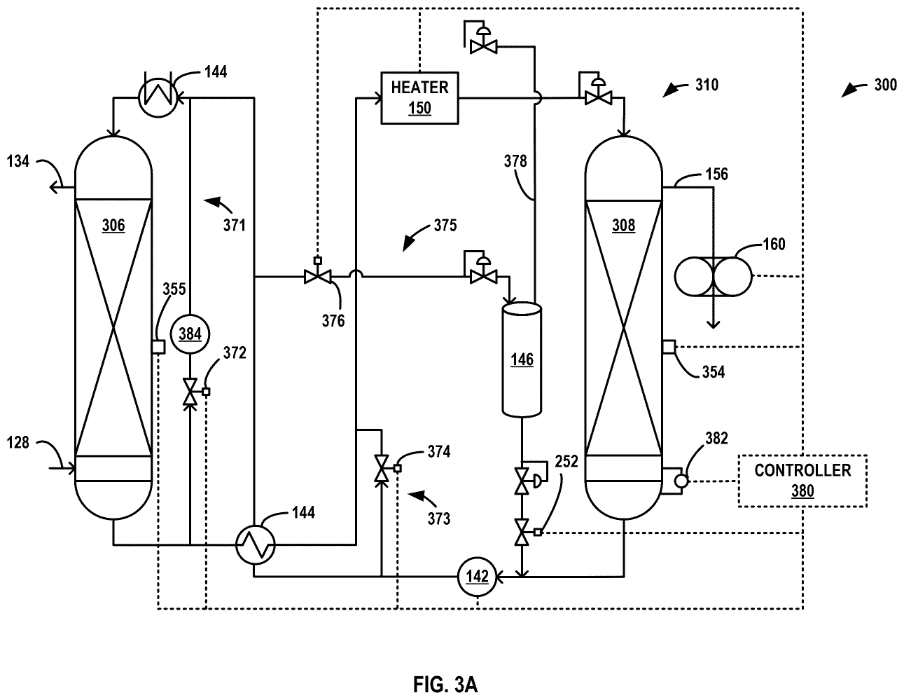

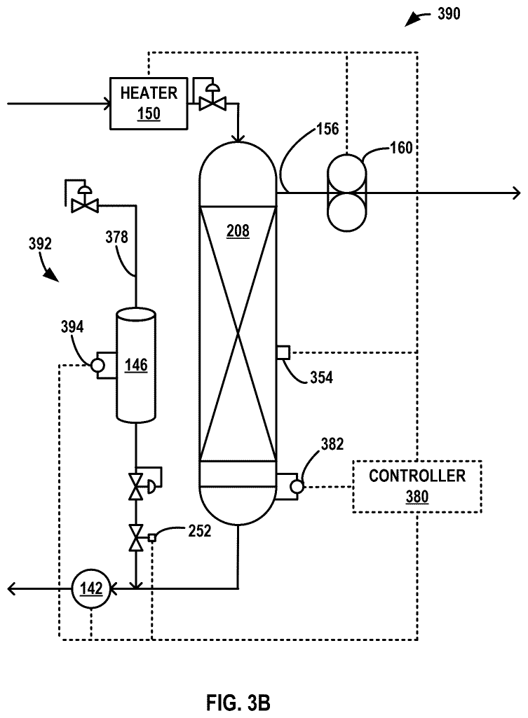

As explained above, packed bed contactors may be capable of operating at high gas flow rates, which may be achieved through recirculation subcircuits, and at deep vacuum, which may be accommodated through pressure management of the liquid sorbent pump. FIG. 3A is a schematic diagram illustrating a liquid sorbent circuit of an example contaminant removal system for removing contaminants using one or more liquid sorbent-based packed bed contactors. Unless otherwise indicated, components of contaminant removal system 300 may be operationally similar to similarly named or suffixed (e.g., 2XX) components of contaminant removal system 200 of FIG. 2.

Liquid sorbent circuit 310 includes various subcircuits configured to control a flow rate and/or pressure to various components within or connected to liquid sorbent circuit 310. In the example of FIG. 3A, liquid sorbent circuit 310 includes a scrubber recirculation subcircuit 371, a stripper recirculation subcircuit 373, and a pump recirculation subcircuit 375.

A desired flow rate of the liquid sorbent through scrubber 306, stripper 308, and liquid sorbent circuit 310 may be different, as scrubber 306 and stripper 308 may absorb and desorb contaminants at different rates, and liquid sorbent circuit 310 may generally benefit from having a lower overall volume, weight, and heating and cooling load. These differences in desired flow rate may be accommodated by operating scrubber recirculation subcircuit 371 at a first flow rate, operating stripper recirculation subcircuit 373 at a second flow rate, and operating a remainder of liquid sorbent circuit 310 at a third flow rate, which may be lower than the first or second flow rates. By recirculating liquid sorbent, an exchange of liquid sorbent between scrubber 306 and stripper 308, and correspondingly an amount heating or cooling applied to the liquid sorbent by components of liquid sorbent circuit 310, may be reduced.

Each of scrubber recirculation subcircuit 371 and stripper recirculation subcircuit 373 is configured to recirculate liquid sorbent through the respective scrubber 306 or stripper 308. Scrubber recirculation subcircuit 371 includes a scrubber recirculation valve 372 and a scrubber recirculation pump 384 configured to control a flow rate of the liquid sorbent bypassing stripper 308 and recirculating back to scrubber 306. Stripper recirculation subcircuit 373 includes a stripper recirculation valve 374 configured to control a flow rate of the liquid sorbent bypassing scrubber 306 and recirculating back to stripper 308.

By enabling independent control of a flow rate of liquid sorbent through each of scrubber 306 and stripper 308, scrubber and stripper recirculation subcircuits 371, 373 may enable greater control over design of scrubber 306 or stripper 308 and/or conditions within scrubber 306 or stripper 308. As one example, stripper 308 and scrubber 306 may have different sizes, as stripper 308 may not handle as high of a gas phase flow as scrubber 306. Additionally, an absence of a stripping gas to stripper 308 may result in reduced mass transfer from the liquid phase to the gas phase, such that by recirculating the liquid sorbent through stripper 308, an effective residence time of the liquid sorbent may be increased, providing additional time for desorption.

As another example, stripper 308 and scrubber 306 may have different types of packing media based a desired pressure drop through the packed bed. Stripper 308 and scrubber 306 may have sufficiently high liquid flow rates through the packed bed to enhance an amount of packing media that receives flow, thereby increasing surface area and mass transfer. Additionally or alternatively, to achieve a deep vacuum in the middle of the packed bed of stripper 308, stripper 308 may include a relatively open packing and large bed.

While operating stripper 308 at a deep vacuum may increase a rate of desorption of contaminants from the liquid sorbent, such deep vacuum may reduce a pressure head at liquid sorbent pump 142. If the pressure head is too low, liquid sorbent pump 142 may cavitate, causing damage and limiting a service life. To maintain the pressure head at liquid sorbent pump 142, liquid sorbent circuit 310 is configured to control a pressure at liquid sorbent pump 142 by controlling pressures and/or liquid levels within liquid sorbent circuit 310 and stripper 308.

In the example of FIG. 3A, liquid sorbent circuit 310 includes pump recirculation subcircuit 375 configured to recirculate liquid sorbent from an outlet of liquid sorbent pump 142 to an inlet of liquid sorbent pump 142 via liquid sorbent storage vessel 146. Pump recirculation subcircuit 375 includes a pump recirculation valve 376 configured to control a flow rate of liquid sorbent bypassing scrubber 306 and stripper 308. In the example of FIG. 3A, liquid sorbent circuit 310 includes sorbent storage pressure assembly 378 configured to control a pressure within liquid sorbent storage vessel 146.

In some examples, the pressure head at liquid sorbent pump 142 may be controlled by maintaining a liquid level in stripper 308 and/or liquid sorbent storage vessel 146. For example, pressure head on liquid sorbent pump 142 may be limited by the presence of gas, such as in a headspace of stripper 308 and/or liquid sorbent storage vessel 146. As such, the liquid level may be maintained sufficiently high to maintain the pressure head at liquid sorbent pump 142. In the example of FIG. 3A, stripper 308 includes a level sensor 382 configured to detect a level of liquid sorbent in stripper 308.

Various other parameters and components of liquid sorbent circuit 310 may be configured to increase a pressure head at liquid sorbent pump 142. For example, liquid sorbent circuit 310 may limit the vacuum to stripper 308 and components downstream of stripper 308, such as through inclusion of pressure regulators and restrictions, and/or may reduce a pressure drop between stripper 308 and liquid sorbent pump 142, such as through larger diameter lines. If possible, stripper 308 and/or liquid sorbent storage vessel 146 may be elevated above liquid sorbent pump 142 to increase a pressure head on liquid sorbent pump 142.

System 300 includes a process control system that includes a controller 380 and one or more sensor sets (not labeled). Controller 380 may include any of a wide range of devices, including control circuitry, processors (e.g., one or more microprocessors, one or more application specific integrated circuits (ASICs), one or more field programmable gate arrays (FPGAs), or the like), processing circuitry, one or more servers, one or more desktop computers, one or more notebook (i.e., laptop) computers, one or more cloud computing clusters, or the like.

Controller 380 may be configured to receive measurements from the one or more sensor sets and/or components of system 300 and/or send control signals to components of system 300, such as indicated by the various dashed lines in FIG. 3A. Controller 380 may be communicatively coupled to and configured to receive measurement signals from the one or more sensor sets, and other process control components (not shown) of contaminant removal system 300, such as: control valves for various streams; pumps; heaters; heat exchangers; compressors; and the like. The sensor sets may include instrumentation configured to detect any of a pressure, temperature, flow rate, and/or contaminant concentration (e.g., carbon dioxide concentration or water concentration) of a liquid or gas stream of contaminant removal system 300. Controller 380 may be configured to use the detected conditions to control operation of system 300, and in some instances, other components of contaminant removal system 300, to function as described in the application.

Controller 380 may be configured to control a concentration of contaminants and a humidity of cabin 102. For example, controller 380 may be configured to receive carbon dioxide and humidity measurements at an inlet and outlet of cabin 102, compare the carbon dioxide and humidity measurements to carbon dioxide and humidity set points, and control various parameters of liquid sorbent circuit 310, such as a flow rate of liquid sorbent via a speed of liquid sorbent pump 142, a temperature of stripper 308 via power to heater 150, a vacuum of stripper 308 via power to compressor (or vacuum pump) 160, an amount of agitation of the liquid sorbent via frequency or power level of ultrasound transducers 354, 355, or an amount of condensed water via cooling fluid flow to condensing heat exchanger 204, to increase or decrease a rate of absorption and/or desorption of carbon dioxide and humidity.

In some examples, to assist in controlling the concentration of contaminants and humidity in cabin 102, controller 380 may be configured to control a flow rate of liquid sorbent to each of scrubber 306 and stripper 308. For example, controller 380 may be configured to control a flow rate of liquid sorbent through scrubber 306 and stripper 308 via scrubber recirculation valve 372 and stripper recirculation valve 374.

Controller 380 may be configured to control a pressure head at liquid sorbent pump 142. For example, controller 380 may be configured to receive a liquid level measurement of stripper 308 from level sensor 382 and/or a vacuum measurement of stripper 308, compare the measurements to thresholds associated with a minimum or desired net positive suction head for liquid sorbent pump 142, and control a level of stripper 308 via operation of pump recirculation valve 374 and/or assembly 378 and/or control vacuum to stripper 308 via power to compressor 160 to maintain the pressure at liquid sorbent pump 142 at or above the minimum or desired net positive suction head to avoid cavitation.

As explained above, a packed bed contactor may be particularly suited for use as a stripper for desorbing contaminants from a liquid sorbent. In some instances, the packed bed contactor may be part of a modular contaminant desorption system that provides a contaminant desorption function and capacity to a liquid sorbent-based contaminant removal system. Such a modular system may closely integrate various components that interact to maintain operation of the packed bed contactor. In contrast to a packed bed contactor used as a scrubber, which may be generally maintained on a gas side at ambient temperature, a packed bed contactor used as a stripper may require careful control of gas side vacuum, temperature, and liquid side pressure to ensure adequate operation.

FIG. 3B is a schematic diagram illustrating an example contaminant desorption system 390 for desorbing contaminants from a liquid sorbent using a liquid sorbent-based packet bed contactor. Contaminant desorption system 390 includes stripper 208 that is a packed bed contactor configured to desorb one or more contaminants from a liquid sorbent. In addition to stripper 208, contaminant desorption system 390 includes various components configured to control parameters related to desorption of contaminants in stripper 208, such as a temperature of the liquid sorbent, a flow rate of the liquid sorbent, and a vacuum within stripper 208. In the example of FIG. 3B, contaminant desorption system 390 includes a liquid sorbent pump 142 configured to receive the liquid sorbent from stripper 208 and a vacuum pump 160 configured to maintain a vacuum on stripper 208 and pressurize a contaminant stream 156 from stripper 208.

Contaminant desorption system 390 may also include a pressure assembly 392 configured to regulate a pressure head at liquid sorbent pump 142. Pressure assembly 392 may be configured to maintain an adequate pressure head at an inlet to liquid sorbent pump 142 by controlling a pressure of the liquid sorbent and/or stripper 208. In the example of FIG. 3B, pressure assembly 392 includes liquid sorbent supply valve 252, sorbent storage pressure assembly 378, and a level sensor 394. Liquid sorbent supply valve 252 may be configured to discharge liquid sorbent from liquid sorbent storage vessel 146 into the liquid sorbent stream upstream of liquid sorbent pump 142. Sorbent storage pressure assembly 378 may be configured to control a pressure within liquid sorbent storage vessel 146. Level sensor 394 may be configured to detect a level of liquid sorbent in liquid sorbent storage vessel 146.

In the example of FIG. 3B, contaminant desorption system includes a controller 380 configured to control operation of the various components of contaminant desorption system. For example, controller 380 may be configured to control vacuum pump 160 to maintain the vacuum on stripper 208, control liquid sorbent pump 142 to discharge the liquid sorbent from stripper 208, control pressure assembly 252, including liquid sorbent supply valve 252 and/or sorbent storage pressure assembly 378, to regulate the pressure head at liquid sorbent pump 142, control a heater 150 to heat the liquid sorbent prior to entry into stripper 208, and/or control an ultrasonic transducer 354 to agitate the liquid sorbent in stripper 208.

Controller 380 may receive feedback from various sensors, such as a flow rate sensor to detect a flow rate of the liquid sorbent, a pressure sensor to detect a vacuum within stripper 208, level sensor 394 to detect a level of liquid sorbent in liquid sorbent storage vessel 146, and/or level sensor 382 to detect a level of liquid sorbent within stripper 208.

Contaminant desorption system 390 may be configured as a modular system to provide a particular contaminant desorption capacity to a liquid sorbent-based contaminant removal system, such as a range of flow rates or residence times of liquid sorbent. While illustrated as including a single stripper 208, in some examples, contaminant desorption system 390 may include multiple strippers 208 coupled to a common liquid sorbent inlet, liquid sorbent outlet, and contaminant outlet headers, such that vacuum pump 160 and/or liquid sorbent pump 142 may be sized according to the capacity of the multiple strippers 208.

The disclosure describes systems and techniques for generating a medium vacuum, such as for desorbing contaminants, such as carbon dioxide, from a liquid sorbent. Such contaminants may be removed at flow rates and pressures that conventional vacuum sources, such as vacuum pumps, may not be well suited for handling due to size or complexity. Contaminant removal systems described herein may be utilized as part of an environmental control system (ECS), such as in watercraft, aircraft, spacecraft, and the like. In some examples, contaminant reduction systems may be used in an ECS of a resource-limited environment, such as a watercraft, having a relatively high personnel load (e.g., greater than about 8 people). Such resource-limited, high personnel environments may be particularly suited for a contaminant removal system that includes components that use low amounts of power and operate with lower complexity, while still maintaining a high throughput for the relatively large crew. In addition to desorbing contaminants, vacuum systems described herein may be used for other applications involving transfer of fluids, such as to provide a driving force for sampling or removing condensed water (e.g., by vaporization and subsequent removal of the water vapor).

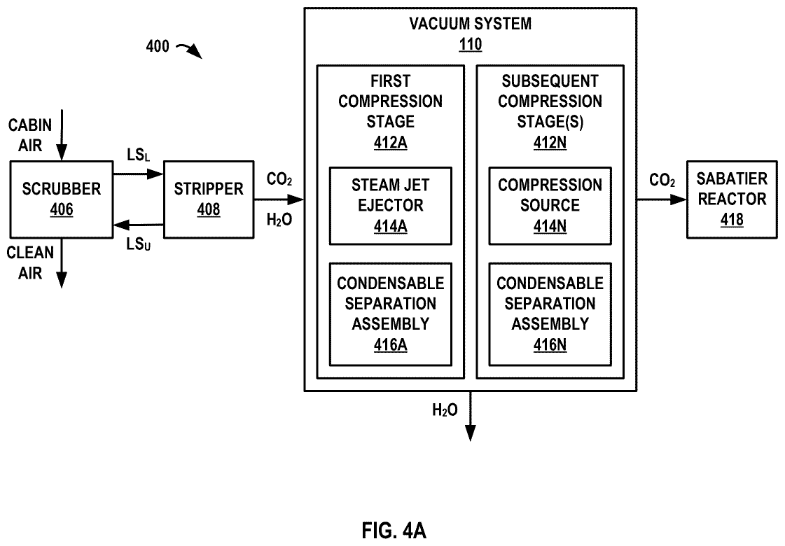

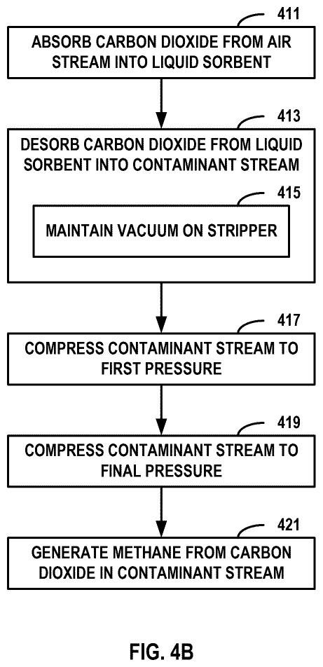

FIG. 4A is a block diagram illustrating an example contaminant removal system 400 for removing contaminants from an air stream. In the example of FIG. 4A, contaminant removal system 400 is configured to remove at least a portion of the contaminants in the air stream using one or more liquid sorbents. A liquid sorbent may include any liquid configured to absorb and desorb a gaseous species. Liquid sorbents may be used with contactors that contact an air stream with or draw an air stream from the liquid sorbent, such as across one or more hydrophobic porous membranes (e.g., membrane contactors) or on a surface of packing media (e.g., packed bed or rotary contactors). While contaminant removal system 400 of FIG. 4A will be described with respect to hollow fiber membrane contactors, in other examples, contaminant removal system 400 may include other contactors that are not membrane contactors, such as packed bed contactors.

Contaminant removal system 400 includes one or more scrubbers 406.

Scrubber 406 is configured to absorb one or more contaminants from an air stream (“CABIN AIR”) into a liquid sorbent. The one or more contaminants include carbon dioxide (CO₂) and other contaminants, such as water vapor (H₂O). Contaminant removal system 400 includes one or more strippers 408 downstream of scrubber 406. Stripper 408 is configured to desorb the one or more contaminants from the liquid sorbent and discharge an air stream (“CLEAN AIR”) having a lower concentration of contaminants than the received air stream. Loaded liquid sorbent (LS_{L}) that includes a relatively high concentration of contaminants is transferred from scrubber 406 to stripper 408, and unloaded liquid sorbent (LS_{U}) that includes a relatively low concentration of contaminants is transferred from stripper 408 to scrubber 406.

Contaminant removal system 400 includes a vacuum system 410. Vacuum system 410 is configured to maintain a medium vacuum on stripper 408, such as less than 50 torr. Drawing such a vacuum on stripper 408 may lower a partial pressure of contaminants on a vapor side of a membrane of stripper 408 and increase a partial pressure gradient of the contaminants across the membrane or other liquid sorbent interface, thereby increasing a rate of desorption.

The contaminant stream desorbed by and discharged from stripper 408 is at a relatively low pressure, and may include condensables, such as water vapor, which may inhibit a downstream process, such as water recovery, compression, a Sabatier reaction, or other chemical reaction. In addition to drawing a vacuum on stripper 408, vacuum system 410 is configured to condition the contaminant stream desorbed from stripper 408, including compressing the contaminant stream to a final pressure for storage or post-processing (e.g., a Sabatier reaction), and separating various condensable gases, such that the contaminant stream includes substantially only non-condensable contaminants such as carbon dioxide. For example, the conditions of the Sabatier reaction, in combination with flow conditions and a composition of a catalyst, may be selected for a relatively high carbon dioxide conversion and methane selectivity. Such conditions may include a temperature between about 250 degrees Celsius (° C.) and about 450° C., and a pressure between about 55 kPa and about 100 kPa.

Vacuum system 410 includes a first compression stage 412A configured to both maintain the vacuum on stripper 408 and compress the contaminant stream received from stripper 408 to a first, intermediate pressure. For small contaminant removal systems, one or more stages of vacuum pumps having a relative low flow rate may be capable of handling the relatively low flow rate of the contaminant stream. However, for larger contaminant removal systems configured to remove contaminants for a greater number of personnel, scaling up the size or number of vacuum pumps may not be feasible due to size, reliability, or complexity.

To maintain both a medium vacuum and a relatively high flow rate of the contaminant stream, first compression stage 412A includes a steam jet ejector 414A. Steam jet ejector 414A is configured to receive both the contaminant stream and a high pressure steam stream, such as from an on-board boiler or other steam source. Steam jet ejector 414A expands the volume of the steam to generate a vacuum on stripper 408 via the contaminant stream and decreases the velocity of the contaminant stream to compresses the contaminant stream.

First compression stage 412A includes a condensable separation assembly 416A configured to remove condensables, such as water, from the contaminant stream. For example, the contaminant stream may include a high proportion of steam from the steam jet ejector, which would otherwise require large amounts of power to compress if not removed prior to subsequent compression stages 412N. Condensable separation assembly 416A may include various equipment, such as a condenser configured to condense steam from steam jet ejector 414A and a water separator configured to separate the condensed water from the contaminant stream.

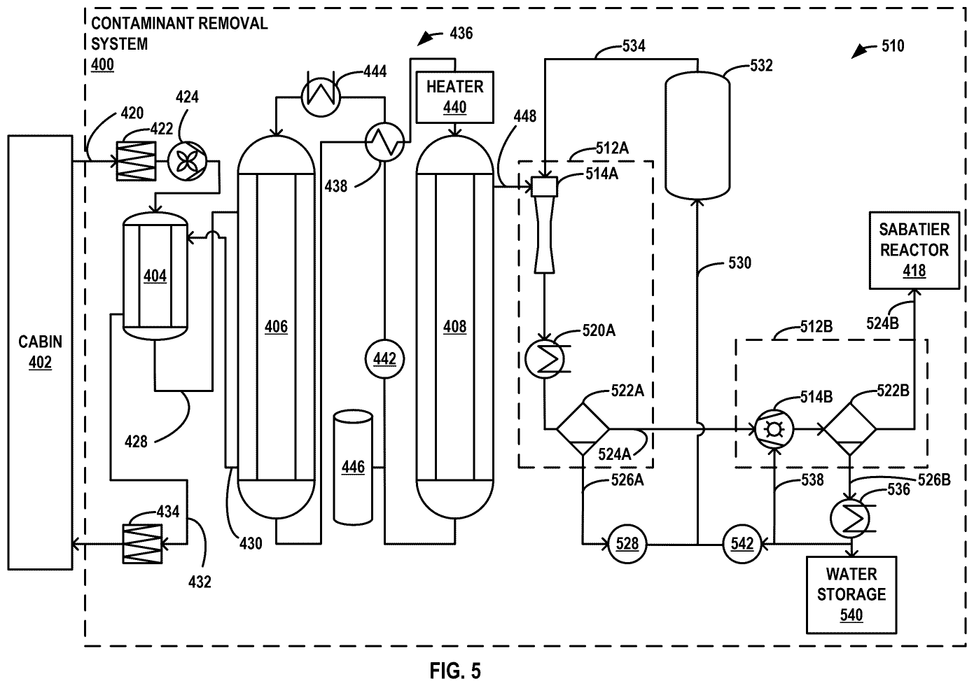

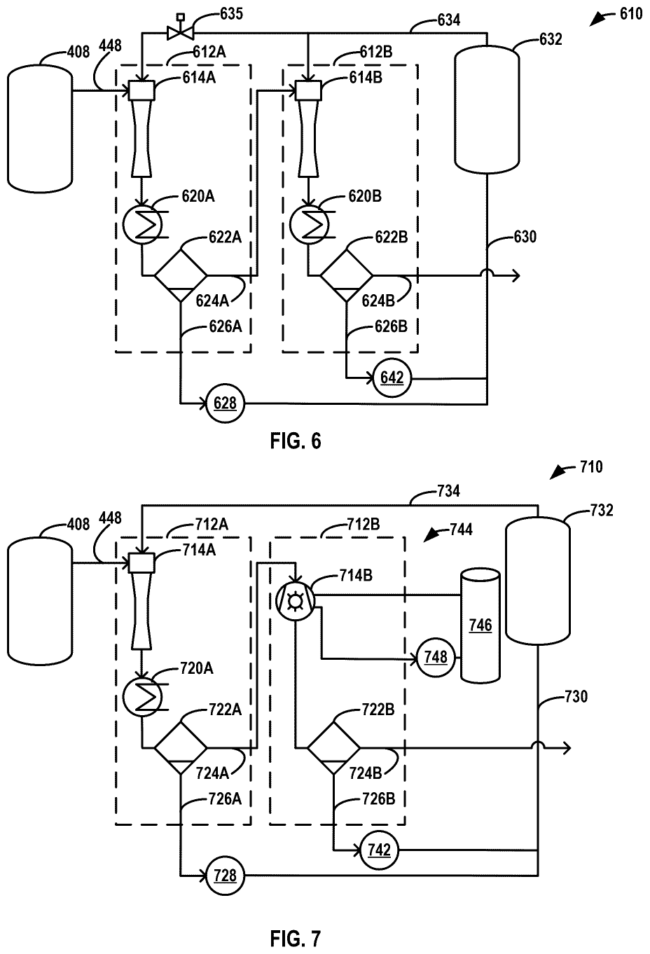

Contaminant removal system 400 includes one or more subsequent compression stages 412N downstream of first compression stage 412A to compress the contaminant stream to a final pressure. Each subsequent compression stage 412N is configured to further compress the fluid stream to an intermediate or final pressure. For example, each subsequent compression stage 412N may include a compression source 414N configured to compress the contaminant stream and a condensable separation assembly 416N configured to separate condensables, such as water, from the contaminant stream. For example, compression source 414N may include an additional steam jet ejector (e.g., as illustrated in FIG. 6), a liquid ring pump, (e.g., as illustrated in FIGS. 5 and 7), or other high flow rate compression source, while condensable separation assembly 416N may include a water separator downstream of compression source 414N and configured to separate water from the contaminant stream.

In the example of FIG. 4A, contaminant removal system 400 includes a Sabatier reactor 418 downstream of vacuum system 410. Sabatier reactor 418 is configured to receive carbon dioxide from vacuum system 410 and hydrogen from a hydrogen source, such as a methane pyrolysis system or oxygen generation system (e.g., an electrolysis system). In some examples, Sabatier reactor 418 may directly receive the contaminant stream from vacuum system 410, such that Sabatier reactor 418 may continuously receive carbon dioxide from stripper 408 via vacuum system 410 while contaminants are desorbed by stripper 408.

By using a steam jet ejector to generate a vacuum on stripper 408, contaminant removal system 400 may operate more reliably and be capable of higher throughput than contaminant removal systems that use vacuum pumps to draw a vacuum and compress a contaminant stream. For example, a single vacuum pump, while capable of reaching a medium vacuum, may be impracticably large and unreliable at high volumetric flow rates, and multiple smaller vacuum pumps arranged in parallel may be difficult and complex to control. In contrast, steam jet ejector 414A may have no or few moving parts, may be capable of handling high flow rates, and may operate with reasonably high pressure ratios (e.g., 1:1.5 to 1:10). Water used to produce the steam may be recycled from other systems, including water removed from the contaminant stream.

FIG. 4B is an example flowchart of a method for compressing a fluid stream, such as a contaminant stream, from a vacuum. FIG. 4B will be described with respect to FIG. 4A; however, other systems may be used to perform the method of FIG. 4B, including for fluid streams other than contaminant streams.

The method of FIG. 4B includes absorbing one or more contaminants from a cabin air stream into a liquid sorbent (411). For example, scrubber 406 of contaminant removal system 400 may be maintained at particular conditions, such as temperature and flow rate of liquid sorbent, to facilitate transfer of the one or more contaminants into the liquid sorbent, such as by controlling a pump to circulate the liquid sorbent between scrubber 406 and stripper 408, controlling a heat exchanger to cool the liquid sorbent prior to entry into scrubber 406, and/or controlling a heat exchanger to recover a portion of heat from liquid sorbent discharged from stripper 408. These particular conditions may be based on a particular measured or anticipated composition of the cabin air stream.

The method of FIG. 4B includes desorbing the one or more contaminants from the liquid sorbent (413). For example, stripper 408 may be maintained at particular conditions, such as temperature and flow rate of liquid sorbent and vacuum from vacuum system 410, to facilitate transfer of the one or more contaminants from the liquid sorbent, such as by controlling steam jet ejector 414A to maintain a vacuum at stripper 408 or controlling a heater to heat the liquid sorbent prior to entry into stripper 408. These particular conditions may be based on a particular measured or anticipated composition of cabin air stream 420. In the example of FIG. 4B, desorbing the carbon dioxide from the liquid sorbent includes maintaining a medium vacuum on stripper 408 (415), such as less than 50 torr. Such medium vacuum may reduce an amount of heating of the liquid sorbent prior to entry into stripper 408.

The method of FIG. 4B includes compressing the contaminant stream to a first, intermediate pressure (417) via first compression stage 412A. For example, steam jet ejector 414A may be operated at particular conditions, such as stream flow rate or pressure, to maintain a vacuum on stripper 408 and compress the contaminant stream to the intermediate pressure. The method of FIG. 4B includes further compressing the contaminant stream to a final pressure (419) via one or more subsequent compression stages 412N. For example, compression source 414N may be operated at particular conditions, such as steam flow rate or pressure for a steam jet ejector or rotary speed for a liquid ring pump, to compress the contaminant stream to a pressure for storage or post-processing, such as about 55 kPa to about 100 kPa for a Sabatier reaction. While not illustrated in the example of FIG. 4B, either of steps 417 and 419 may include separating condensables, such as water, from the contaminant stream, via condensable separation assemblies 416A and 416N.

The method of FIG. 4B includes generating methane from carbon dioxide in the contaminant stream (421). For example, Sabatier reactor 418 may be configured to receive the contaminant stream from vacuum system 410 and hydrogen gas from another source, and generate methane from the carbon dioxide and the hydrogen gas.

FIG. 5 is a schematic diagram illustrating an example contaminant removal system 400, such as contaminant removal system 400 of FIG. 4A, that includes a vacuum system for desorbing and compressing contaminants. Contaminant removal system 400 includes a cabin air circuit (not labeled) configured to circulate cabin air between a cabin 402 and scrubber 406 via a membrane dehumidifier 404. In the example of FIG. 5, a cabin air stream 420 includes a filter 422 configured to remove particulates from cabin air stream 420 prior to entry into scrubber 406 and a blower 424 configured to draw cabin air into scrubber 406, while clean air stream 432 includes a filter 434 configured to remove any leaked liquid sorbent and/or further filter clean air from clean air stream 432 prior to entry into cabin 402.

In the example of FIG. 5, contaminant removal system 400 includes membrane dehumidifier 404 to capture humidity from cabin air stream 420 to recover humidity into clean air stream 432 that may otherwise be absorbed by the liquid sorbent at scrubber 406. For example, membrane dehumidifier 404 may be positioned between cabin 402 and scrubber 406, such that cabin air received by scrubber 406 may include a lower humidity than cabin air received by contaminant removal system 400 from cabin 402. On one side, membrane dehumidifier 404 may be configured to receive cabin air stream 420 as a feed gas stream and discharge cabin air in a dehumidified air stream 428 to scrubber 406 having a lower humidity. On an opposite side, membrane dehumidifier 404 may be configured to receive a dehumidified clean air stream 430 from scrubber 406 and discharge clean air to clean air stream 432 having a higher humidity. By capturing humidity from cabin air prior to entry of the cabin air from cabin air stream 420 into scrubber 406, a greater amount of humidity may be preserved and/or a reduced amount of water may be removed by stripper 408 through evaporative cooling.

In the example of FIG. 5, contaminant removal system 400 is configured to remove at least a portion of the contaminants in dehumidified air stream 428 using one or more liquid sorbents. A liquid sorbent may include any liquid configured to absorb and desorb a gaseous species. Liquid sorbents may be water soluble, hygroscopic (i.e., capable of absorbing moisture from the air), capable of absorbing or desorbing contaminants in response to a change in solubility driven by a change in temperature, and/or capable of releasing water by evaporation, such as by elevating the temperature or reducing the water partial pressure in the gas phase surrounding the liquid sorbent. In some examples, the liquid sorbent may be an ionic liquid sorbent. Ionic liquid sorbents may be salts that are generally comprised of an anion and an organic cation. These salts may be liquid at their temperature of use, have effectively zero vapor pressure, be generally nontoxic, and/or have sufficient stability to resist deterioration. In some examples, ionic liquid sorbents may contain relatively large organic cations and any of a variety of anions, which may be tailored to obtain desired characteristics, such as characteristics that improve absorption of the particular contaminant under operating conditions of contaminant removal system 400. A variety of ionic liquid sorbents may be used including, but not limited to, imidazolium salts, such as 1-ethyl-3-methylimidazolium (EMIM) acetate (Ac).

In contaminant removal system 400, the liquid sorbent is dissolved in water to form a liquid sorbent mixture. A concentration of liquid sorbent in the liquid sorbent mixture may be sufficiently high to remove a particular or set of contaminants and sufficiently low that the liquid sorbent remains in solution through operating ranges (e.g., temperature range, pH range) and/or maintains a low viscosity for maintaining high mass transfer. In some examples, the liquid sorbent mixture may further include a dissolved promoter. The promoter may be configured to increase a rate of removal of a contaminant, such as water or carbon dioxide, from an air stream. For example, the promoter may be configured to reduce a viscosity of the liquid sorbent, change a pH of the liquid sorbent, increase a thermal stability of the liquid sorbent, increase a capacity of the liquid sorbent for the contaminant, or increase an absorption rate of the contaminant into the liquid sorbent. Absorption of the contaminants by the liquid sorbent may be determined by a concentration of the contaminants in the corresponding air stream. Liquid sorbents may be used with membrane contactors that contact an air stream with or draw an air stream from the liquid sorbent across one or more hydrophobic porous membranes.

Scrubbers 406 and/or strippers 408 described herein may include one or more membrane separators configured to flow air on a first side and flow liquid sorbent on a second, opposite side. For example, a membrane separator may include a plurality of parallel membrane contactors. A membrane contactor may include a cylindrical module filled with parallel or woven hollow porous fibers forming a hydrophobic porous membrane. For example, dimensions of these hollow fibers could be less than about 3 mm, and the pore dimension could be less than about 2 microns. The high surface area of the hollow fiber membrane contactors enables a high mass transfer of contaminant gases, such as carbon dioxide and water, into the respective liquid sorbent using a relatively small system volume and weight. The material of the hollow fibers can be selected such that the liquid sorbent does not wet the pores, and the trans-membrane pressure is kept sufficiently low to prevent pore penetration. As a result, the membrane contactor may ensure that the liquid sorbent and gas stream do not need further separation, such that contaminant removal system 400 may act in an orientation-independent way. Fiber materials may include, but are not limited to, hydrophobic materials such as polypropylene, polyvinylidene fluoride, polysulfone, polyimide, polytetrafluoroethylene (PTFE), and the like. In some examples, a coating may be applied to reduce liquid flow through the pores. Coatings that may be used include, but are not limited to, PTFE, a crosslinked siloxane, perfluorinated polymers, functionalized nanoparticles, and the like to prevent liquid flow through the pores.

While described in FIG. 5 as flowing through a “tube” side, liquid sorbent flow can be either on the “tube” side or the “shell” side, while gas is flowed on the opposite side.

Scrubber 406 is configured to absorb one or more contaminants from cabin air stream 420 into the liquid sorbent and discharge a clean air stream 432 to cabin 402. Clean air stream 432 has a lower concentration of contaminants than cabin air stream 420. For example, clean air stream 432 may have a concentration of carbon dioxide that is about 25% to about 99% less than a concentration of carbon dioxide in cabin air stream 420. Scrubber 406 includes one or more separation membranes, each configured to flow (e.g., provide or direct flow of) cabin air from cabin air stream 420 on a gas phase side (e.g., a tube side) of the respective membrane and flow the liquid sorbent on a liquid phase side (e.g., a shell side) of the membrane.

On a gas phase side, scrubber 406 is configured to receive cabin air from cabin air stream 420 that includes contaminants from cabin 402. Contaminants may pass through the membrane due to a concentration gradient between the cabin air and the liquid sorbent and become absorbed by the liquid sorbent, while the liquid sorbent may not substantially flow through the membrane. As a result, clean air from clean air stream 432 discharged from scrubber 406 may have a lower concentration of contaminants than cabin air from cabin air stream 420 received by scrubber 406.

Scrubber 406 is configured to discharge clean air stream 432 to cabin 402 via membrane dehumidifier 404. On a liquid phase side, scrubber 406 is configured to receive unloaded liquid sorbent, such as from a liquid sorbent storage 446. The unloaded liquid sorbent may flow through scrubber 406 and absorb carbon dioxide and other gaseous contaminants from cabin air through the membrane(s) of scrubber 406. As a result, the loaded liquid sorbent discharged from scrubber 406 may have a higher concentration of contaminants than the unloaded liquid sorbent received by scrubber 406. Scrubber 406 may discharge the loaded liquid sorbent containing the contaminants to stripper 408.

Stripper 408 is configured to desorb the contaminants, including carbon dioxide, from the liquid sorbent into contaminant stream 448. Stripper 408 includes one or more separation membranes, each configured to collect (e.g., receive through the membrane) contaminants on a gas phase side (e.g., a tube side) of the respective membrane and flow the loaded liquid sorbent on a liquid phase side (e.g., a shell side) of the membrane. On a liquid phase side, stripper 408 is configured to receive loaded liquid sorbent from scrubber 406 and desorb contaminants from the loaded liquid sorbent. Contaminants may flow across fibers of the membrane due to a concentration gradient, while the liquid sorbent may not substantially flow across the fibers of the membrane. As a result, unloaded liquid sorbent discharged from stripper 408 may have a lower concentration of contaminants than the loaded liquid sorbent received by stripper 408. On a gas phase side, stripper 408 is configured to discharge the contaminants in contaminant stream 448. Contaminant stream 448 may be continuously removed from stripper 408 to assist migration of the contaminants from the loaded liquid sorbent into contaminant stream 448.

While scrubber 406 and stripper 408 have been described with respect to membrane contactors, in some examples, either of scrubber 406 and/or stripper 408 may include another type of contactor configured to absorb or desorb gases from a liquid sorbent, such as a packed bed contactor.

Contaminant removal system 400 includes liquid sorbent circuit 436 configured to circulate liquid sorbent between scrubber 406 and stripper 408. For example, a pump 442 may pump unloaded liquid sorbent from stripper 408 into scrubber 406. Unloaded liquid sorbent may include unused liquid sorbent free of contaminants or regenerated liquid sorbent having a lower concentration of contaminants than the loaded liquid sorbent. Liquid sorbent storage 446 may store liquid sorbent, such as in a relatively cool state.

Liquid sorbent circuit 436 may include one or more components for controlling a temperature of the liquid sorbent. In some examples, the unloaded liquid sorbent may be cooled by a regenerative heat exchanger 438 and/or a heat exchanger 444 prior to entry into scrubber 406. Heat exchanger 438 is configured to exchange heat between a relatively hot unloaded liquid sorbent from stripper 408 and a relatively cool loaded liquid sorbent from scrubber 406. Heat exchanger 444 configured to receive the unloaded liquid sorbent from stripper 408, cool the unloaded liquid sorbent, and discharge the cooled, unloaded liquid sorbent to scrubber 406.

Liquid sorbent circuit 436 may include one or more heaters 440 upstream of stripper 408. Heaters 440 may be configured to heat the liquid sorbent prior to entry into stripper 408 to increase desorption of contaminants from the liquid sorbent.

Contaminant removal system 400 includes a vacuum system 510. Vacuum system 510 is configured to pressurize (or “compress”) contaminant stream 448 and remove condensables, such as water, from contaminant stream 448. For example, for carbon dioxide removed from stripper 408 to be reacted efficiently by Sabatier reactor 418, vacuum system 510 may pressurize contaminant stream 448 to a moderate pressure and remove nearly all water from contaminant stream 448. However, this pressure may be substantially higher than a pressure at which stripper 408 would otherwise be operated at. In some examples, vacuum system 510 is configured to pressurize the carbon dioxide to a reaction pressure. For example, vacuum system 510 may be configured to compress contaminant stream 448 from less than or equal to about 10 torr up to a pressure between about 50 kPa and about 100 kPa.

Vacuum system 510 includes multiple compression stages that, individually or collectively, draw a vacuum on stripper 408 to aid desorption of contaminants from the liquid sorbent and compress contaminant stream 448 from the vacuum to a final pressure, such as a pressure used for a Sabatier reaction. In the example of FIG. 5, vacuum system 510 includes two compression stages; however, vacuum system 510 may include additional compression stages, such as for higher flow rates of contaminants or higher final pressures. Vacuum system 510 includes a first compression stage 512A and a second compression stage 512B downstream of first compression stage 512A.

While both first compression stage 512A and second compression stage 512B are configured to compress a fluid stream, first compression stage 512A is specifically configured to maintain a medium vacuum (e.g., less than or equal to about 50 torr) on stripper 408. To maintain a medium vacuum on stripper 108, first compression stage 512A includes a steam jet ejector 514A. Steam jet ejector 514A is fluidically coupled to stripper 408 via contaminant stream 448 and fluidically coupled to a steam source via a high pressure steam stream 534. High pressure steam stream 534 has a substantially higher pressure than stripper 408. In the example of FIG. 5, vacuum system 510 includes a boiler 532 as a steam source configured to generate steam.

However, in other examples, the steam source may be another steam source, such as a high pressure steam line used for other systems.

Steam jet ejector 514A is configured to both maintain a medium vacuum on stripper 408 and compress contaminant stream 448 received from stripper 408 from the vacuum to a first pressure. Steam jet ejector 514A may be particularly suited for a resource-limited environment, as steam jet ejector 514A leverages the high velocity of the steam to both create a vacuum on stripper 408 and compress contaminants from contaminant stream 448 in a single, continuous process without moving parts.

Steam jet ejector 514A includes a converging-diverging nozzle, a low-pressure region, and a diffuser. Steam jet ejector 514A may receive high pressure steam from steam stream 534 and discharge the high pressure steam through the converging-diverging nozzle, causing the steam to rapidly expand and accelerate and converting the thermal energy of the steam into kinetic energy. As the steam exits the nozzle at high velocity, it creates the low-pressure region at the nozzle exit that has a lower pressure than stripper 408. The low-pressure region causes contaminants in the liquid sorbent to be drawn into ejector 514A as contaminant stream 448, which is subsequently entrained by the high-velocity steam. Contaminant stream 448 mixes with the high-velocity steam in the mixing chamber of ejector 514A, and the kinetic energy of the steam is transferred to contaminant stream 448, causing contaminant stream 448 to accelerate. The mixture of steam and process gas then enters the diffuser of ejector 514A, where the velocity decreases, and the pressure increases due to the conversion of kinetic energy back into pressure energy. This compression raises the pressure of contaminants to a level higher than the intermediate pressure in stripper 408, but typically lower than the pressure of steam stream 534. Steam jet ejector 514A may generally operate based on a compression ratio from about 1:1.5 to about 1:10. In some instances, a vacuum pump may pull an initial vacuum on stripper 408 prior to beginning operation of steam jet ejector 514A.

In addition to components for compressing contaminant stream 448, first compression stage 512A includes components for removing condensables, such as water, to reduce an energy load on subsequent compression stages, such as second compression stage 512B. For example, contaminant stream 448 is a mixed stream that includes a condensable fluid, such as water, and a non-condensable fluid, such as carbon dioxide. For carbon dioxide removed from stripper 408 to be reacted efficiently by Sabatier reactor 418, vacuum system 510 may pressurize contaminant stream 448 to a moderate pressure and remove nearly all water from contaminant stream 448. In the example of FIG. 5, compression stages that include a steam jet ejector, such as first compression stage 512A, includes a condenser 520A configured to condense water, including a large proportion of steam from steam jet ejector 514A, and a water separator 522A configured to separate the condensed water from the compressed contaminant stream 448.

Condenser 520A is configured to condense desorbed water from contaminant stream 448. Condenser 520A may be configured to cool contaminant stream 448 and condense water from contaminant stream 448. For example, condenser 520A may be coupled to a cooling medium system or other cooling system that circulates a cooling medium to cool contaminant stream 448. A variety of condensers may be used for condenser 520A including, but not limited to, shell and tube heat exchangers, plate-fin, surface coolers, heat pipes, thermoelectric devices, cooling jackets, and the like. Water separator 522A is positioned downstream of condenser 520A and is configured to separate desorbed water from the carbon dioxide. Water separator 522A may be configured to remove water from contaminant stream 448, discharge an intermediate compressed stream 524A to second compression stage 512B, and discharge water condensate stream 526A to a pump 528. A variety of water separators may be used for water separator 522A including, but not limited to, static phase separators, capillary phase separator, membrane phase separators, centrifugal/rotary separators, and the like.

In some examples, at least a portion of water condensate stream 526A, including condensed steam, may be recycled for use as high pressure stream. In the example of FIG. 5, pump 528 is configured to pump condensed water back to boiler 532 via a water return stream 530, such that boiler 532 may generate steam from the condensed water. For example, after mixing with steam, contaminant stream 448 may have a relatively high proportion of water, such as greater than about 60 volume percent. This water can be condensed and returned to produce steam, thereby limiting this volume of water in intermediate compressed stream 524A to a small portion relative to contaminant stream 448.

Second compression stage 512B is configured to receive intermediate compressed stream 524A at the intermediate pressure and further compress stream 524A to a final pressure. To compress stream 524A, second compression stage 512B includes a liquid ring pump 514B. Liquid ring pump 514B is configured to receive intermediate compressed stream 524A at the intermediate pressure and compress stream 524A to the final pressure as final compressed stream 524B. Liquid ring pump 514B may be well suited for handling wet gases and vapors with significant amounts of liquid carryover, such as entrained water, without incurring damage. Liquid ring pump 514B may also be capable of maintaining a consistent vacuum level and handling varying gas volumes without significant changes in performance. Liquid ring pump 514B may also have greater reliability and quieter operation than, for example, a rotary vane vacuum pump, as liquid ring pump 514B may have fewer moving parts producing less vibration and mechanical wear and tear. Liquid ring pump 514B may include one or more stages for operating at various pressure ratios including, but not limited to, a single-stage liquid ring pump (e.g., for a pressure ratio of 1.5:1 to 3:1), a two-stage liquid ring pump (e.g., for a pressure ratio of 3:1 to 10:1), or other liquid ring pumps.

Liquid ring pumps may work best at relatively higher pressures where the sealing fluid does not evaporate. Liquid ring pumps may be particularly suited for streams that include a condensable fluid, as dry vacuum pumps struggle to handle condensing fluids in the pump. For example, dry vacuum pumps will typically handle condensable fluids by including a ballast gas or sending some of the compressed gas back around to the inlet. A ballast gas dilutes the product stream, which may be undesirable in resource limited environments or for product streams that are purified for further reaction. Sending some of the compressed gas back around to the inlet reduces the percentage of the stream that is condensable, limiting condensation, but also has a limit, as it increases the size of the stream being compressed so increases pump size, weight, and power.

Like first compression stage 512A, second compression stage 512B may include components for removing condensables, such as water, to reduce a water content and increase a purity of final compressed stream 524B. In the example of FIG. 5, compression stages that include a liquid ring pump, such as second compression stage 512B, includes a water separator 522B downstream of liquid ring pump 514B and configured to separate water from contaminant stream 448. Water separator 522B may be configured to remove water from contaminant stream 448, discharge final compressed stream 524B from vacuum system 510, such as to Sabatier reactor 418, and discharge water condensate stream 526B through a chiller 536 to either a pump 542 or water storage 540.

In some examples, liquid ring pump 514B may utilize removed water, such as from water condensate stream 526B, as a sealing fluid. For example, liquid ring pump 514B may use water or an aqueous medium as a sealing fluid. During vacuum operation, the low pressure within liquid ring pump 514B and/or heat absorbed by liquid ring pump 514B may cause evaporation of the sealing fluid. To compensate for this evaporation, chilled water from chiller 536 may be supplied to liquid ring pump 514B through a sealing fluid stream 538.

In the example of FIG. 5, contaminant removal system 400 may include one or more systems or components configured to further process final compressed stream 524B. Contaminant removal system 400 includes Sabatier reactor 418 configured to generate hydrocarbons using carbon dioxide removed by scrubber 406. Sabatier reactor 418 may require a water concentration of less than 10% to react hydrogen with carbon dioxide. Sabatier reactor 418 may be configured to generate methane from the carbon dioxide and, to a lesser degree, carbon monoxide. Sabatier reactor 418 may include a catalyst configured to increase a reaction rate of the Sabatier reaction. In some examples, a structure of the catalyst may be configured to further increase thermal and/or mass transfer of reactants and products. For example, Sabatier reactor 418 may include a catalyst in the form of any of a mesh, a packed bed, a microchannel grid, or other structure or combination of structures having a high surface area, high thermal conductivity, and/or high reactant throughput. Catalysts that may be used include, but are not limited to, nickel, ruthenium, rhodium, and the like, alone or on a support, such as aluminum oxide.

In some examples, Sabatier reactor 418 is configured to operate at a relatively high carbon dioxide conversion (e.g., >70%) and methane selectivity (e.g., >80%) at relatively low temperatures and pressures. For example, the catalyst may be configured to achieve such high carbon dioxide conversion and methane selectivity at temperatures less than about 450° C. and/or pressures less than about 100 kPa. A pressure within Sabatier reactor 418 may be sufficient to condense water downstream of Sabatier reactor 418 upon cooling from the reaction temperature within the reactor so that water is not lost downstream, and/or may increase an efficiency of the reactions in Sabatier reactor 418 to increase yield and/or permit small volume, such as a pressure greater than about 50 kPa. On the other hand, maintaining a pressure at less than 100 kPa may create a vacuum in Sabatier reactor 418, such that methane or other combustibles may not leak out of Sabatier reactor 418 into a cabin space. Further, maintaining a low pressure may reduce an amount of power used by vacuum system 510.

Contaminant removal system 400 may include a control system (not shown) communicatively coupled to and configured to receive measurement signals from one or more sensor sets, and other process control components (not shown) of contaminant removal system 400, such as: control valves for cabin air stream 420, clean air stream 432, contaminant stream 448, intermediate compressed stream 524A, final compressed stream 524B, and inlets/outlets to heat exchangers 438 and 444, heater 440, liquid sorbent storage 446; pumps 442, 528, and 542; blower 424; liquid ring pump 514B; boiler 532; and the like. The control system is configured to control a concentration of one or more contaminants within the environment of cabin 402. For example, the control system may be configured to receive a concentration measurement for a contaminant, such as carbon dioxide, such as from a cabin air sensor set or a carbon dioxide concentration sensor in cabin 402. The control system may be configured to determine whether the concentration measurement of the contaminant exceeds a concentration setpoint. For example, the concentration setpoint may be a target concentration of the contaminant for maintaining cabin 402 below a threshold contaminant concentration. The control system may be configured to send, in response to the concentration measurement of the contaminant exceeding the concentration setpoint, a control signal to decrease a concentration of the contaminant in an air stream returned to cabin 402. For example, the control system may send a control signal to control a flow rate of the liquid sorbent mixture; a temperature of the liquid sorbent mixture at scrubber 406 or stripper 408; a flow rate of the cabin air stream from cabin 402; a vacuum at stripper 408; or any other variable that may control a rate of removal of the contaminant from the cabin air stream from cabin 402 and/or a rate of desorption of the contaminant from the liquid sorbent.

Vacuum system 510 of FIG. 5 has been described with respect to a second compression stage 512B having liquid ring pump 514B supplied with water as a sealing fluid. Such a system may be particularly suited for high reliability and high utilization of limited resources (e.g., water). However, in other examples, other compression sources may be used to compress an intermediate compressed stream and/or other sealing fluids may be used to seal a liquid ring pump.

FIG. 6 is a schematic diagram illustrating an example vacuum system 610 for contaminant desorption that includes a steam jet ejector 614A in a first compression stage 612A and a steam jet ejector 614B in a second compression stage 612B. Unless otherwise indicated, components of vacuum system 610 may correspond to similarly named and suffixed (e.g., 5XX) components of vacuum system 510 of FIG. 5.

Steam jet ejector 614A is configured to compress contaminant stream 448 from stripper 408 to a first pressure and maintain a vacuum on stripper 408 less than about 50 torr. Steam jet ejector 614A may receive high pressure steam from steam stream 634. A pressure of the steam may be reduced from a pressure of steam exiting a boiler 632, such as via a pressure reducing valve 635. A condenser 620A is configured to condense water from contaminant stream 448, including steam introduced via steam jet ejector 614A, and a water separator 622A configured to separate the condensed water from contaminants in contaminant stream 448 and discharge a water condensate stream 626A.

Steam jet ejector 614B is configured to compress intermediate compressed stream 624A from first compression stage 612A to a final pressure. Steam jet ejector 614B may receive high pressure steam from steam stream 634. A condenser 620B is configured to condense water from intermediate compressed stream 624A, including steam introduced via steam jet ejector 614B, and a water separator 622B configured to separate the condensed water from contaminants in intermediate compressed stream 624A and discharge a water condensate stream 626B. As a result, second compression stage 612B may output a final compressed stream 624B that is at a desired pressure and high purity of contaminants.

Vacuum system 610 may be configured to recycle condensed water from water condensate streams 626A and 626B for generating steam. A pump 628 and a pump 642 may supply water from respective condensate streams 626A and 626B to boiler 632 via a water supply stream 630. A pressure of high pressure steam may be delivered to each steam jet ejector 614A or 614B to maintain a desired pressure ratio. For example, boiler 632 may be operated to produce high pressure steam for steam jet ejector 614B, and a pressure and/or flow rate of the high pressure steam may be reduced via reducing valve 635 to produce lower pressure and/or flow rate steam for steam jet ejector 614A than for steam jet ejector 614B.

FIG. 7 is a schematic diagram illustrating an example vacuum system 710 for contaminant desorption that includes a steam jet ejector 714A in a first compression stage 712A and a liquid ring pump 714B, sealed by an ionic liquid, in a second compression stage 712B. Unless otherwise indicated, components of vacuum system 710 may correspond to similarly suffixed (e.g., 5XX) components of vacuum system 510 of FIG. 5.

Steam jet ejector 714A is configured to compress contaminant stream 448 from stripper 408 to a first pressure and maintain a vacuum on stripper 408 less than about 50 torr. Steam jet ejector 714A may receive high pressure steam from boiler 732 via a steam stream 734. A condenser 720A is configured to condense water from contaminant stream 448, including steam introduced via steam jet ejector 714A, and a water separator 722A configured to separate the condensed water from contaminants in contaminant stream 448 and discharge a water condensate stream 726A.

Liquid ring pump 714B is configured to compress intermediate compressed stream 724A from first compression stage 712A to a final pressure. A water separator 722B is configured to separate the water from contaminants in intermediate compressed stream 724A and discharge a water condensate stream 726B. As a result, second compression stage 712B may output a final compressed stream 724B that is at a desired pressure and high purity of contaminants.

Due to a relatively low pressure of intermediate compressed stream 724A received by liquid ring pump 714B, a sealing liquid used to seal liquid ring pump 714B may be subject to evaporation loses if the sealing liquid has a high vapor pressure. To reduce an amount of sealing liquid that evaporates, the sealing liquid may include an ionic liquid having a low vapor pressure. Vacuum system 710 further includes an ionic liquid circuit 744 configured to circulate the ionic liquid between liquid ring pump 714B and an ionic liquid storage 746 via an ionic liquid pump 748.

In some examples, the sealing liquid may be subject to absorption additions if the gases are condensed in liquid ring pump 714B. For example, if condensable gases such as water are condensed in liquid ring pump 714B, the condensables will combine with the sealing fluid and need to be removed. If the condensable is immiscible with the sealing fluid, the condensable can be separated such as by draining water or sealing fluid from the bottom of a vessel, or vice versa from the top. If the condensable and sealing fluid are miscible, ionic liquid circuit 744 may include means to remove the condensable, such as via heating, pulling vacuum in a vessel headspace, stripping via a hollow fiber membrane or packed bed, or any other mechanism for separating miscible fluids.

In some examples, the ionic liquid used to seal liquid ring pump 714B is hygroscopic. A hygroscopic ionic liquid may assist in pulling water from intermediate compressed stream 724A, which reduce an amount of water condensed downstream. However, such absorption may require the water to be removed from the ionic liquid as that water accumulates. In some examples, the ionic liquid used to seal liquid ring pump 714B is hydrophobic. A hydrophobic ionic liquid may resist pulling water from intermediate compressed stream 724A, which may reduce an amount of water that may be removed the ionic liquid.

Vacuum system 710 may be configured to recycle condensed water from water condensate streams 726A and 726B for generating steam, maintaining a water balance of the ionic liquid of liquid ring pump 714B (not shown), and/or maintaining a water balance of the liquid sorbent. A pump 728 and a pump 742 may supply water from respective condensate streams 726A and 726B to boiler 732 via a water supply stream 730.

Example 1: A contaminant removal system includes a scrubber configured to absorb one or more contaminants from an air stream into a liquid sorbent; a stripper configured to desorb the one or more contaminants from the liquid sorbent; a liquid sorbent circuit configured to circulate the liquid sorbent between the scrubber and the stripper; and a conditioning assembly configured to maintain a vacuum on the stripper, wherein at least the stripper comprises a packed bed contactor.

Example 2: The contaminant removal system of example 1, wherein the liquid sorbent circuit comprises at least one recirculation subcircuit configured to recirculate a portion of the liquid sorbent through the packed bed contactor.

Example 3: The contaminant removal system of any of examples 1 and 2, wherein the scrubber comprises a packed bed contactor.

Example 4: The contaminant removal system of any of examples 1 through 3, wherein the liquid sorbent circuit comprises: a liquid sorbent pump configured to receive the liquid sorbent from the stripper and a liquid sorbent vessel; and a sorbent storage pressure assembly configured to control a pressure of the liquid sorbent vessel to regulate a pressure head at the liquid sorbent pump.

Example 5: The contaminant removal system of any of examples 1 through 4, wherein the packed bed contactor includes at least one ultrasonic transducer configured to agitate the liquid sorbent.

Example 6: The contaminant removal system of any of examples 1 through 5, further comprising a condensing heat exchanger configured to dehumidify the air stream prior to discharging the air stream to the scrubber.

Example 7: The contaminant removal system of example 6, wherein the scrubber is configured to discharge a clean air stream, and wherein the contaminant removal system further comprises a humidifier configured to rehumidify the clean air stream using water removed by the condensing heat exchanger.

Example 8: The contaminant removal system of example 7, wherein the humidifier comprises at least one of a membrane humidifier or an evaporator.

Example 9: The contaminant removal system of any of examples 1 through 8, wherein the conditioning assembly comprises: a vacuum pump configured to maintain a vacuum on the stripper and pressurize a contaminant stream from the stripper; and a water separator configured to remove water from the contaminant stream.

Example 10: The contaminant removal system of any of examples 1 through 9, wherein the one or more contaminants include carbon dioxide, and wherein the contaminant removal system further comprises a Sabatier reactor configured to generate methane from the carbon dioxide.

Example 11: A contaminant desorption system includes a stripper configured to desorb one or more contaminants from a liquid sorbent; a liquid sorbent pump configured to receive the liquid sorbent from the stripper; and a vacuum pump configured to maintain a vacuum on the stripper and pressurize a contaminant stream from the stripper, wherein the stripper comprises a packed bed contactor.

Example 12: The contaminant desorption system of example 11, further comprising a pressure assembly configured to regulate a pressure head at the liquid sorbent pump.

Example 13: The contaminant desorption system of example 12, further includes control the vacuum pump to maintain the vacuum on the stripper; control the liquid sorbent pump to discharge the liquid sorbent from the stripper; and control the pressure assembly to regulate the pressure head at the liquid sorbent pump.

Example 14: A method for removing one or more contaminants from an air stream includes absorbing, by a scrubber, the one or more contaminants from the air stream into a liquid sorbent; desorbing, by a stripper, the one or more contaminants from the liquid sorbent; circulating, by a liquid sorbent circuit, the liquid sorbent between the scrubber and the stripper; and maintaining, by a conditioning assembly, a vacuum on the stripper, wherein at least the stripper comprises a packed bed contactor.

Example 15: The method of example 14, further comprising recirculating, by at least one recirculation subcircuit of the liquid sorbent circuit, a portion of the liquid sorbent through the packed bed contactor.

Example 16: The method of any of examples 14 and 15, wherein circulating the liquid sorbent further comprises: receiving, by a liquid sorbent pump, the liquid sorbent from the stripper and a liquid sorbent vessel; and controlling, by a sorbent storage pressure assembly, a pressure of the liquid sorbent vessel to regulate a pressure head at the liquid sorbent pump.

Example 17: The method of any of examples 14 through 16, further comprising agitating, by at least one ultrasonic transducer, the liquid sorbent in the packed bed contactor.

Example 18: The method of any of examples 14 through 17, further comprising dehumidifying, by a condensing heat exchanger, the air stream prior to discharging the air stream to the scrubber.

Example 19: The method of example 18, further includes discharging, by the scrubber, a clean air stream, and rehumidifying, by a humidifier, the clean air stream using water removed by the condensing heat exchanger.

Example 20: The method of any of examples 14 through 19, wherein the one or more contaminants include carbon dioxide, and wherein the method further includes generating, by a Sabatier reactor, methane from the carbon dioxide.

Example 21: A vacuum system includes a first compression stage includes compress a fluid stream received from a vessel to a first pressure; and maintain a vacuum on the vessel less than about 50 torr; and one or more subsequent compression stages downstream of the first compression stage, wherein each subsequent compression stage is configured to further compress the fluid stream from the first pressure to a final pressure.

Example 22: The vacuum system of example 21, wherein the first stage further comprises: a condenser configured to condense one or more condensables from the fluid stream and steam from the steam jet ejector; and a water separator configured to separate the condensed steam from the fluid stream.

Example 23: The vacuum system of example 22, further comprising a boiler configured to generate steam from the condensed steam.

Example 24: The vacuum system of any of examples 21 through 23, wherein at least one subsequent stage comprises an additional steam jet ejector.

Example 25: The vacuum system of example 24, wherein each subsequent stage includes a condenser configured to condense steam from the additional steam jet ejector; and a water separator configured to separate the condensed water from the fluid stream.

Example 26: The vacuum system of any of examples 21 through 25, wherein at least one subsequent stage comprises a liquid ring pump.

Example 27: The vacuum system of example 26, wherein the liquid ring pump is sealed using an ionic liquid.

Example 28: The vacuum system of example 27, wherein the ionic liquid used to seal the liquid ring pump is hygroscopic.

Example 29: The vacuum system of any of examples 27 and 28, wherein the ionic liquid used to seal the liquid ring pump is hydrophobic.

Example 30: The vacuum system of any of examples 26 through 29, wherein each subsequent stage that comprises the liquid ring pump comprises a water separator downstream of the liquid ring pump and configured to separate water from the fluid stream.

Example 31: The vacuum system of any of examples 21 through 30, wherein the fluid stream comprises a mixed stream that includes at least one condensable fluid and at least one non-condensable fluid.

Example 32: A contaminant removal system includes a scrubber configured to absorb one or more contaminants from an air stream using a liquid sorbent; a stripper configured to desorb the one or more contaminants from the liquid sorbent into a contaminant stream; and the vacuum system of any of claims 1 to 10, wherein the fluid stream comprises the contaminant stream.

Example 33: The contaminant removal system of example 32, further comprising a Sabatier reactor configured to receive the compressed contaminant stream.

Example 34: The contaminant removal system of any of examples 32 and 33, wherein at least one of the scrubber or the stripper comprises at least one hollow fiber membrane contactor.

Example 35: A method for compressing a fluid stream from a vacuum includes receiving, by a first compression stage, the fluid stream from a vessel, wherein the first compression stage comprises a steam jet ejector; maintaining, by the first compression stage, the vacuum on the vessel less than about 50 torr; compressing, by the first compression stage, the fluid stream to a first pressure; and compressing, by one or more subsequent compression stages downstream of the first compression stage, the fluid stream to a final pressure.

Example 36: The method of example 35, wherein the first stage further comprises: condensing, by a condenser, steam from the steam jet ejector; and separating, by a water separator, the condensed steam from the fluid stream.

Example 37: The method of example 36, further comprising, generating, by a boiler, steam from the condensed steam.

Example 38: The method of any of examples 35 through 37, wherein the fluid stream received from the vessel comprises a mixed stream that includes a condensable fluid.

Example 39: The method of any of examples 35 through 38, wherein the fluid stream comprises one or more contaminants, and wherein the vessel comprises a stripper configured to desorb the one or more contaminants from a liquid sorbent.

Example 40: The method of any of examples 35 through 39, wherein the final pressure is greater than about 55 kPa.

Various examples have been described. These and other examples are within the scope of the following claims.

What is claimed is:

1. A vacuum system, comprising:
a first compression stage comprising a steam jet ejector, wherein the first compression stage is configured to:
compress a fluid stream received from a vessel to a first pressure; and
maintain a vacuum on the vessel less than about 50 torr; and
one or more subsequent compression stages downstream of the first compression stage, wherein each subsequent compression stage is configured to further compress the fluid stream from the first pressure to a final pressure.

2. The vacuum system of claim 1, wherein the first stage further comprises:
a condenser configured to condense one or more condensables from the fluid stream and steam from the steam jet ejector; and
a water separator configured to separate the condensed steam from the fluid stream.

3. The vacuum system of claim 2, further comprising a boiler configured to generate steam from the condensed steam.

4. The vacuum system of claim 1, wherein at least one subsequent stage comprises an additional steam jet ejector.

5. The vacuum system of claim 4, wherein each subsequent stage comprising an additional steam jet ejector further comprises:
a condenser configured to condense steam from the additional steam jet ejector; and
a water separator configured to separate the condensed water from the fluid stream.

6. The vacuum system of claim 1, wherein at least one subsequent stage comprises a liquid ring pump.

7. The vacuum system of claim 6, wherein the liquid ring pump is sealed using an ionic liquid.

8. The vacuum system of claim 7, wherein the ionic liquid used to seal the liquid ring pump is hygroscopic.

9. The vacuum system of claim 7, wherein the ionic liquid used to seal the liquid ring pump is hydrophobic.

10. The vacuum system of claim 6, wherein each subsequent stage that comprises the liquid ring pump comprises a water separator downstream of the liquid ring pump and configured to separate water from the fluid stream.

11. The vacuum system of claim 1, wherein the fluid stream comprises a mixed stream that includes at least one condensable fluid and at least one non-condensable fluid.

12. A contaminant removal system, comprising:
a scrubber configured to absorb one or more contaminants from an air stream using a liquid sorbent;
a stripper configured to desorb the one or more contaminants from the liquid sorbent into a contaminant stream; and
the vacuum system of any of claims 1 to 10, wherein the fluid stream comprises the contaminant stream.

13. The contaminant removal system of claim 12, further comprising a Sabatier reactor configured to receive the compressed contaminant stream.

14. The contaminant removal system of claim 12, wherein at least one of the scrubber or the stripper comprises at least one hollow fiber membrane contactor.

15. A method for compressing a fluid stream from a vacuum, comprising:
receiving, by a first compression stage, the fluid stream from a vessel, wherein the first compression stage comprises a steam jet ejector;
maintaining, by the first compression stage, the vacuum on the vessel less than about 50 torr;
compressing, by the first compression stage, the fluid stream to a first pressure; and
compressing, by one or more subsequent compression stages downstream of the first compression stage, the fluid stream to a final pressure.

16. The method of claim 15, wherein the first stage further comprises:
condensing, by a condenser, steam from the steam jet ejector; and
separating, by a water separator, the condensed steam from the fluid stream.

17. The method of claim 16, further comprising, generating, by a boiler, steam from the condensed steam.

18. The method of claim 15, wherein the fluid stream received from the vessel comprises a mixed stream that includes a condensable fluid.

19. The method of claim 15,
wherein the fluid stream comprises one or more contaminants, and
wherein the vessel comprises a stripper configured to desorb the one or more contaminants from a liquid sorbent.

20. The method of claim 15, wherein the final pressure is greater than about 55 kPa.
